(19) United States
(12) Patent Application Publication
(10) Pub. No.: US 2024/0173389 A1
(43) Pub. Date: May 30, 2024

(54) Compositions and Methods For Reducing Immune Intolerance

(71) Applicant: LAPIX THERAPEUTICS, INC., Boston, MA (US)

(72) Inventors: Anas Fathallah, Wakefield, MA (US); Abdulraouf Ramadan, Malden, MA (US)

(73) Assignee: LAPIX THERAPEUTICS, INC., Boston, MA (US)

(21) Appl. No.: 18/279,612

(22) PCT Filed: Mar. 11, 2022
(86) PCT No.: PCT/US2022/071082

§ 371 Date: Aug. 31, 2023

Related U.S. Application Data
(60) Provisional application No. 63/248,086, filed on Sep. 24, 2021.
(60) Provisional application No. 63/159,667, filed on Mar. 11, 2021.

(51) Int. Cl. A61K 39/00; A61K 9/00; A61K 9/127; A61K 31/198; A61K 31/661; A61K 45/06; A61P 37/06

(52) U.S. Cl. CPC A61K 39/0008; A61K 9/0019; A61K 9/127; A61K 31/198; A61K 31/661; A61K 45/06; A61P 37/06; A61K 2039/55555

(57) ABSTRACT

Provided herein are compositions comprising amino acids, e.g., hydroxyamino acids, thioamino acids, or pharmaceutically acceptable salts thereof, that can be used to reduce immune intolerance in a subject. The compositions can be used, for example, to treat autoimmune disorders or in combination with an antigenic therapy, such as a protein or gene therapy, to improve the efficacy of the antigenic therapy.

### RELATED APPLICATIONS

This application claims the benefit of U.S. Provisional Application No. 63/159,667, filed on Mar. 11, 2021, and U.S. Provisional Application No. 63/248,086, filed on Sep. 24, 2021. The entire teachings of the above applications are incorporated herein by reference.

### BACKGROUND

Enzyme and protein replacement therapy is a successful therapeutic strategy for treating congenital disorders where an endogenous protein is mutated, missing, or otherwise aberrant. However, clinical administration of foreign enzyme or protein is associated with the development of unwanted immune response toward the enzyme or protein. The unwanted immune response could lead to neutralization of the enzyme/protein, or alteration of its pharmacokinetics. In many circumstances, patients do not have alternative therapeutic options, making the unwanted immune response to therapy a major issue facing enzyme and protein replacement therapy recipients.

Similarly, gene therapy offers a promising approach to treat a number of congenital disorders and other diseases. Immunogenicity of the carrier and/or the genetic material carried within is a major challenge to the clinical application of gene therapy. Existing anti-carrier antibodies is a counter-indication to treatment with some approved gene therapies. Furthermore, nascent anti-carrier antibodies can prevent repeat dosing in subjects that receive the first dose of a gene therapy.

Autoimmune disorders are a collection of disorders in which the body lacks or loses tolerance to self-antigens. This results in the body's immune system attacking healthy cells, and can have debilitating and devastating effects. Current approaches to treating autoimmune disorders rely on general immune suppression at the humoral, cellular and/or complement level, rendering patients immunocompromised and susceptible to opportunistic infections.

Accordingly, there is a need for compositions that can reduce immune intolerance to exogenous antigens (e.g., enzyme replacement therapy, gene therapy) or endogenous antigens (e.g., self-antigens causing autoimmune disorders), for example, by mitigating the immunogenicity of enzyme and protein replacement therapy and/or gene therapy, or increasing self-tolerance to self-antigens.

### SUMMARY

The technology described herein relates to tolerance induction for exogenous antigens (e.g., antigen-specific and/or antigen-exclusive tolerance induction), or for self-antigens. The technology is based on engaging and modulating (e.g., activating) the T-cell immunoglobulin mucin protein (TIM) family of receptors.

Provided herein are methods of immunotolerizing a subject in need thereof to an antigen in a subject. The methods comprise administering to the subject a therapeutically effective amount of a compound of the disclosure, e.g., in the form of a composition described herein.

Also provided herein are methods of inhibiting or reducing an antigen-specific antibody titer in a subject. The methods comprise administering to the subject a therapeutically effective amount of a compound of the disclosure, e.g., in the form of a composition described herein.

Also provided herein are methods of inducing a population of regulatory T-cells in a subject and/or increasing the activity or level of tolerogenic T-cells in a subject. The methods comprise administering to the subject a therapeutically effective amount of a compound of the disclosure, e.g., in the form of a composition described herein.

Also provided herein is a method of treating an autoimmune disorder in a subject in need thereof, comprising administering to the subject a therapeutically effective amount of a compound of the disclosure, e.g., in the form of a composition described herein.

Also provided herein is a method of treating a disease, disorder or condition in a subject in need thereof with an antigenic therapy, comprising administering to the subject the antigenic therapy and a compound of the disclosure, e.g., in the form of a composition described herein. The composition is administered to the subject in an amount sufficient to immunotolerize the subject to the antigenic therapy.

Also provided herein is a composition (e.g., pharmaceutical composition) comprising a compound of the disclosure for a use described herein (e.g., treatment of an autoimmune disorder; treatment of a disease, disorder or condition treatable with antigenic therapy). Also provided herein is use of a composition comprising a compound of the disclosure for the manufacture of a medicament for a use described herein (e.g., treatment of an autoimmune disorder; treatment of a disease, disorder or condition treatable with antigenic therapy).

### BRIEF DESCRIPTION OF THE DRAWINGS

The foregoing will be apparent from the following more particular description of example embodiments.

FIG. 1A shows the results of anti-FVIII titer analysis conducted at the end of the treatment period by treatment group for the study described in Example 1.

FIG. 1B shows the results of anti-FVIII titer analysis after each re-challenge compared to end of treatment by treatment group for the study described in Example 1.

FIG. 1C shows the results of FoxP3 positive T-cell proliferation at the end of the treatment period by treatment group for the study described in Example 1.

FIG. 2A shows the results of anti-GAA titer analysis conducted at the end of the treatment period by treatment group for the study described in Example 2.

FIG. 2B shows the results of anti-GAA titer analysis after each re-challenge compared to end of treatment by treatment group for the study described in Example 2.

FIG. 3A shows the results of FoxP3 positive T-cell proliferation at the end of the treatment period by treatment group for the study described in Example 3.

FIG. 3B shows the results of anti-AAV9 titer analysis conducted at the end of the treatment period by treatment group for the study described in Example 3.

FIG. 4 shows the results of FoxP3 positive T-cell proliferation at the end of the treatment period by treatment group for the study described in Example 1.

FIG. 5A shows the dose-response of TNF-α after exposure to different doses of O-phospho-L-serine (OPLS) and O-phospho-L-tyrosine (OPLT).

FIG. 5B shows the dose-response of TNF-α after exposure to different doses of liposomal 1,2-dimyristoyl-sn-glycero-3-phosphocholine (DMPC), liposomal phosphatidyl serine (PS) liposomes, liposomal O-benzyl-L-serine (LOBLS), liposomal S-phenyl-L-cysteine (LSPLC), liposomal S-benzyl-L-cysteine (LSBLC) and liposomal O-benzyl-L-tyrosine (LOBLT).

FIG. 5C shows the dose response of TNF-α after exposure to different doses of DMPC, PS and liposomal and non-liposomal OBLS, SPLC, SBLC and OBLT.

FIG. 5D shows the dose response of TNF-α and TGF-β after exposure to different doses of PS and LOBLT.

FIG. 5E shows the dose response of TNF-α and TGF-β after exposure to different doses of OPLS, OPLT and L-homocysteic acid (LHA).

FIG. 6A shows changes in TNF-αin response to increasing doses of PGN by treatment group.

FIG. 6B shows changes in TNF-α in response to increasing doses of Poly I:C by treatment group.

FIG. 6C shows changes in TNF-α in response to increasing doses of LPS by treatment group.

FIG. 6D shows changes in TNF-α in response to increasing doses of PolyU by treatment group.

FIG. 6E shows changes in TNF-α in response to increasing doses of ODN CPG by treatment group.

FIG. 7A shows the results of FoxP3 positive/CD4 positive T-cell proliferation for O-tert-butyl-L-tyrosine, O-methyl-L-tyrosine, L-tyrosine and L-phenylalanine in the assay described in Example 7.

FIG. 7B shows the results of FoxP3 positive/CD4 positive T-cell proliferation for L-methionine sulfate, 4-nitro-L-phenylalanine, L-cysteine-S-sulfate and trans-4-hydroxy-L-proline in the assay described in Example 7.

### DETAILED DESCRIPTION

A description of example embodiments follows.

### Definitions

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

When introducing elements disclosed herein, the articles “a,” “an,” “the,” and “said” are intended to mean that there are one or more of the elements. Further, the one or more elements may be the same or different.

“Amino acid” refers to a molecule containing an amino acid backbone covalently bonded to an amine group, a carboxylic acid group and an amino acid side chain, which varies between different amino acids. When specified (e.g., at the point of use herein), substitutions and replacements in an amino acid (e.g., the backbone of an amino acid described herein), such as substitution or replacement of a hydroxyl group and/or substitution or replacement of a thiol group in a referenced amino acid, are encompassed in “amino acid”. Thus, for example, recitation of “naturally-occurring amino acid,” without more, refers to the naturally-occurring amino acid unmodified by substitution or replacement. An amino acid can, in some embodiments, be represented by the formula NH₂—CHR^{a}—COOH, wherein R^{a} is the side chain. “Amino acid” includes both naturally-occurring amino acids and non-naturally-occurring amino acids. “Amino acid” also includes canonical amino acids, non-canonical amino acids, L-amino acids and D-amino acids. In one embodiment, an amino acid is a naturally-occurring amino acid. In one embodiment, an amino acid is a L-amino acid.

“Naturally-occurring amino acid” means an amino acid, as that term is used herein, wherein the amino acid side chain corresponds to the side chain of a naturally-occurring amino acid (e.g., naturally occurring in proteins, naturally occurring in nature). A naturally-occurring amino acid can, in some embodiments, be represented by the formula NH₂—CHR^{a}—COOH, wherein R^{a} is the side chain of a naturally-occurring amino acid (e.g., naturally occurring in proteins, naturally occurring in nature), such as an amino acid listed or named in the Table of Selected Naturally Occurring Amino Acids below. Homocysteine, which is a byproduct of methionine metabolism, is also a naturally-occurring amino acid, as is hydroxyproline. “Naturally-occurring amino acid” includes both the D- and L-configurations of the reference amino acid. In one embodiment, a naturally-occurring amino acid is a naturally-occurring L-amino acid.

Table of Common Naturally Occurring Amino Acids

| | Amino acid | Three letter code | One letter code |
|---|---|---|---|
| Hydrophobic | glycine | Gly | G |
| | alanine | Ala | A |
| | isoleucine | Ile | I |
| | leucine | Leu | L |
| | methionine | Met | M |
| | phenylalanine | Phe | F |
| | proline | Pro | P |
| | tryptophan | Trp | W |
| | valine | Val | V |
| Hydrophilic | asparagine | Asn | N |
| | cysteine | Cys | C |
| | glutamine | Gln | Q |
| | serine | Ser | S |
| | threonine | Thr | T |
| | tyrosine | Tyr | Y |
| | glutamic acid | Glu | E |
| | arginine | Arg | R |
| | aspartic acid | Asp | D |
| | histidine | His | H |
| | lysine | Lys | K |
| | sarcosine | Sar | |

“Non-natural amino acid” means an amino acid for which there is no nucleic acid codon. Examples of non-natural amino acids include natural a-amino acids with non-natural side chains, b-amino acids (e.g., b-alanine) and g-amino acids (e.g., g-aminobutyric acid).

“Hydroxyamino acid” refers to an amino acid wherein the amino acid side chain comprises a hydroxyl group. “Hydroxyamino acid” includes both naturally-occurring hydroxyamino acids and non-naturally-occurring hydroxyamino acids. “Hydroxyamino acid” also includes canonical hydroxyamino acids, non-canonical hydroxyamino acids, L-hydroxyamino acids and D-hydroxyamino acids. In one embodiment, a hydroxyamino acid is a naturally-occurring hydroxyamino acid. In one embodiment, a hydroxyamino acid is a L-hydroxyamino acid. Examples of hydroxyamino acids include serine, threonine and tyrosine. A further example of a hydroxyamino acid is hydroxyproline.

“Thioamino acid” refers to an amino acid wherein the amino acid side chain comprises a thiol group. “Thioamino acid” includes both naturally-occurring thioamino acids and non-naturally-occurring thioamino acids. “Thioamino acid” also includes canonical thioamino acids, non-canonical thioamino acids, L-thioamino acids and D-thioamino acids. In one embodiment, a thioamino acid is a naturally-occurring thioamino acid. In one embodiment, a thioamino acid is a L-thioamino acid. Examples of thioamino acids include cysteine and homocysteine.

“Alkyl” refers to a branched or straight-chain, monovalent, hydrocarbon radical having the specified number of carbon atoms. Thus, “(C₁-C₈)alkyl” refers to a radical having from 1-8 carbon atoms in a branched or linear arrangement. Typically, alkyl is (C₁-C₂₅)alkyl, e.g., (C₁-C₁₅)alkyl, (C₁-C₁₀)alkyl, (C₁-C₈)alkyl, (C₂-C₈)alkyl, (C₁-C₆)alkyl, (C₂-C₆)alkyl, (C₁-C₅)alkyl or (C₂-C₅)alkyl. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like. In some aspects, alkyl is optionally substituted, e.g., with one or more substituents described herein.

“Alkenyl” refers to a branched or straight-chain, monovalent, hydrocarbon radical having at least one carbon-carbon double bond and the specified number of carbon atoms. Thus, “(C₂-C₈)alkenyl” refers to a radical having at least one carbon-carbon double bond and from 2-8 carbon atoms in a branched or linear arrangement. Typically, alkenyl is (C₁-C₂₅)alkenyl, e.g., (C₁-C₁₅)alkenyl, (C₁-C₁₀)alkenyl, (C₁-C₈)alkenyl, (C₂-C₈)alkenyl, (C₁-C₆)alkenyl, (C₂-C₆)alkenyl, (C₁-C₅)alkenyl or (C₂-C₅)alkenyl. Examples of alkenyl groups include ethenyl, 2-propenyl, 1-propenyl, 2-methyl-1-propenyl, 1-butenyl, 2-butenyl, 1-pentenyl, 2-pentenyl, 3-pentenyl, allyl, 1,3-butadienyl, 1,3-dipentenyl, 1,4-dipentenyl, 1-hexenyl, 1,3-hexenyl, 1,4-hexenyl, 1,3,5-trihexenyl, 2,4-dihexenyl, and the like. In some aspects, alkenyl is optionally substituted, e.g., with one or more substituents described herein.

“Aryl” refers to a monocyclic or polycyclic (e.g., bicyclic, tricyclic), aromatic, hydrocarbon ring system having the specified number of ring atoms, and includes aromatic rings fused to non-aromatic rings, as long as one of the fused rings is an aromatic hydrocarbon. Thus, “(C₆-C₁₅)aryl” refers to a ring system having from 6-15 ring atoms. Examples of aryl include phenyl, naphthyl and fluorenyl. In some aspects, aryl is optionally substituted, e.g., with one or more substituents described herein.

“Heteroaryl” refers to a monocyclic or polycyclic (e.g., bicyclic, tricyclic), aromatic, hydrocarbon ring system having the specified number of ring atoms, wherein at least one carbon atom in the ring system has been replaced with a heteroatom selected from nitrogen, sulfur and oxygen. Thus, “(C₅-C₁₅)heteroaryl” refers to a heteroaromatic ring system having from 5-15 ring atoms consisting of carbon, nitrogen, sulfur and oxygen. “Heteroaryl” includes heteroaromatic rings fused to non-aromatic rings, as long as one of the fused rings is a heteroaromatic hydrocarbon. A heteroaryl can contain 1, 2, 3 or 4 (e.g., 1, 2 or 3) heteroatoms independently selected from nitrogen, sulfur and oxygen. Typically, heteroaryl is (C₅-C₂₀)heteroaryl, e.g., (C₅-C₁₅)heteroaryl, (C₅-C₁₂)heteroaryl, C₅ heteroaryl or C6 heteroaryl. Monocyclic heteroaryls include, but are not limited to, furan, oxazole, thiophene, triazole, triazene, thiadiazole, oxadiazole, imidazole, isothiazole, isoxazole, pyrazole, pyridazine, pyridine, pyrazine, pyrimidine, pyrrole, tetrazole and thiazole. Bicyclic heteroaryls include, but are not limited to, indolizine, indole, isoindole, indazole, benzimidazole, benzofuran, benzothiazole, purine, quinoline, isoquinoline, cinnoline, phthalazine, quinazoline, quinoxaline, naphthyridine and pteridine. In some aspects, heteroaryl is optionally substituted, e.g., with one or more substituents described herein.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Halogen” and “halo” are used interchangeably herein and each refers to fluorine, chlorine, bromine, or iodine. In some aspects, halo is fluoro, chloro or bromo. In some aspects, halo is fluoro.

“Haloalkyl” includes mono, poly, and perhaloalkyl groups, wherein each halogen is independently selected from fluorine, chlorine, bromine and iodine (e.g., fluorine, chlorine and bromine), and alkyl is as described herein. In one aspect, haloalkyl is perhaloalkyl (e.g., perfluoroalkyl). Examples of haloalkyl include, but are not limited to, trifluoromethyl and pentafluoroethyl.

“Haloalkoxy” refers to a haloalkyl radical attached through an oxygen linking atom, wherein haloalkyl is as described herein. Examples of haloalkoxy include, but are not limited to, trifluoromethoxy.

“Hydroxy” and “hydroxyl” mean —OH.

“Thio” and “thiol” mean —SH.

The term “substituted” refers to replacement of a hydrogen atom with a suitable substituent. Typically, the suitable substituent replaces a hydrogen atom bound to a carbon atom, but a substituent may also replace a hydrogen bound to a heteroatom, such as a nitrogen, oxygen or sulfur atom. It will be understood that “substitution” or “substituted with” includes the implicit proviso that such substitution is in accordance with permitted valence of the substituted atom. It is also preferred that the substituent, and the substitution, result in a stable compound, e.g., which does not spontaneously undergo transformation such as by rearrangement, cyclization, elimination, etc. Suitable substituents for use herein include acyclic and cyclic, branched and unbranched, carbocyclic and heterocyclic, aromatic and non-aromatic substituents of organic compounds. For example, suitable substituents can include halogen, hydroxyl, carbonyl (such as carboxyl, alkoxycarbonyl, formyl, or acyl), thiocarbonyl (such as thioester, thioacetate, or thioformate), alkyl, alkoxy, alkylthio, acyloxy, phosphoryl, phosphate, phosphonate, amino, amido, amidine, imine, cyano, nitro, azido, sulfhydryl, alkylthio, sulfate, sulfonate, sulfamoyl, sulfonamido, sulfonyl, cycloalkyl, heterocyclyl, aralkyl, aryl or heteroaryl. It will be understood by those skilled in the art that substituents can themselves be substituted, if appropriate. Accordingly, substituents can further include an acetamide, for example.

The permissible substituents can be one or more and the same or different for appropriate organic compounds. Thus, an “optionally substituted” group is, in some aspects, substituted with 0-5 (e.g., 0-3, 0, 1, 2, 3, 4, 5) substituents independently selected from halo, (C₁-C₆)alkoxy, (C₁-C₆)haloalkoxy, (C₁-C₆)alkyl, (C₁-C₆)haloalkyl, (C₆-C₁₅)aryl or (C₅-C₁₅)heteroaryl. In some aspects, an optionally substituted aryl or heteroaryl is substituted with 0-5 (e.g., 0-3, 0, 1, 2, 3, 4, 5) substituents independently selected from halo, (C₁-C₆)alkoxy, (C₁-C₆)haloalkoxy, (C₁-C₆)alkyl or (C₁-C₆)haloalkyl. In some aspects, an optionally substituted” aryl or heteroaryl is substituted with 0-5 (e.g., 0-3, 0, 1, 2, 3, 4, 5) substituents independently selected from halo, (C₁-C₃)alkoxy, (C₁-C₃)haloalkoxy, (C₁-C₃)alkyl or (C₁-C₃)haloalkyl. In some aspects, an optionally substituted alkyl or alkenyl is substituted with 0-5 (e.g., 0-3, 0, 1, 2, 3, 4, 5) substituents independently selected from halo (e.g., fluoro), (C₁-C₆)alkoxy, (C₁-C₆)haloalkoxy (e.g., (C₁-C₆)fluoroalkoxy), (C₆-C₁₅)aryl or (C₅-C₁₅)heteroaryl.

The term “optionally substituted”, as used herein, means that substitution is optional and, therefore, it is possible for the atom or moiety designated as “optionally substituted” to be unsubstituted or substituted. Unless otherwise indicated, e.g., as with the terms “substituted” or “optionally substituted,” a group designated herein is unsubstituted.

An indicated moiety (e.g., functional group, such as thiol; atom, such as hydrogen) is “replaced” herein when a suitable substituent takes the place of the indicated moiety in the referenced compound. Thus, for example, when the thiol of homocysteine is replaced with SO₃H, the resulting compound is homocysteic acid.

As used herein, the term “compound of the disclosure” refers to an amino acid described herein, as well as isomers, such as stereoisomers (including diastereoisomers, enantiomers and racemates) and tautomers thereof, isotopically labeled variants thereof (including those with deuterium substitutions), and inherently formed moieties (e.g., polymorphs and/or solvates, such as hydrates) thereof. When a moiety is present that is capable of forming a salt, then salts are included as well, in particular, pharmaceutically acceptable salts thereof.

Compounds of the disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers (including cis and trans double bond isomers), conformational isomers (including rotamers and atropisomers), tautomers) and intermediate mixtures, with all possible isomers and mixtures thereof being included, unless otherwise indicated.

When a disclosed compound is depicted by structure without indicating the stereochemistry, and the compound has one or more chiral centers, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s). When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” can be used to indicate the absolute configuration of substituents around one or more chiral carbon atoms. D- and L- can also be used to designate stereochemistry.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. An enantiomer may be present in an ee of at least or about 50%, about 60%, about 70%, about 80%, about 90%, about 95%, about 98%, about 99% or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 … )}{D 1 + ( D 2 + D 3 + D 4 … )} \vert \times 100 \% ,$$

where D1 and (D2+D3+D4 . . . )

represent the respective fractions of each diastereomer in a mixture, such that D1+D2+D3+D4 . . . ) 32 1. A diastereomer may be present in a de of at least or about 50%, about 60%, about 70%, about 80%, about 90%, about 95%, about 98%, about 99% or about 99.9%.

Unless otherwise stated, compounds of the disclosure include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. In all provided structures, any hydrogen atom can also be independently selected from deuterium (²H), tritium (³H) and/or fluorine (¹⁸F). Such compounds are useful, for example, as analytical tools, as probes in biological assays, or as therapeutic agents in accordance with the present invention.

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺ ((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as “solvates” or “hydrates.” A “hydrate” is a compound that exists in a composition with one or more water molecules. A hydrate can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Antigen,” as used herein, refers to any substance that can be recognized by the immune system. “Antigen” broadly encompasses proteins, such as enzymes, peptides, such as polypeptides, carbohydrates, such as polysaccharides, haptens, nucleic acids and grafts. An antigen can be a self-antigen, an antigen produced, under normal conditions or as part of a disorder, by the body, or a foreign antigen, a non-self-antigen. Examples of self-antigens include self-antigens associated with autoimmune disorders, including any of the self-antigens described herein. Examples of foreign antigens include antigenic therapies (e.g., therapeutic proteins, gene therapies, cellular therapies), allergens and alloantigens.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., induction of immune tolerance, treatment, healing, inhibition or amelioration of physiological response or condition (e.g., unwanted immune response to an antigenic therapy), etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some aspects, a subject is a human. Compounds

A first embodiment is an amino acid (e.g., in some aspects, a naturally-occurring amino acid and/or an L-amino acid), or a pharmaceutically acceptable salt thereof, wherein:
the amino acid is a hydroxyamino acid or thioamino acid;
the hydroxyl of the hydroxyamino acid or the thiol of the thioamino acid, respectively, is substituted with -L(R)ₙ;
L is absent, or a (C₁-C₈)alkyl or (C₂-C₈)alkenyl optionally substituted with one or more fluoro;
each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2.

In some aspects of the first embodiment, the amino acid is serine, threonine, cysteine, homocysteine or tyrosine.

In some aspects of the first embodiment, L is absent or (C₁-C₃)alkyl (e.g., in some aspects, (C₁)alkyl) optionally substituted with one or more fluoro.

In some aspects of the first embodiment, each R is independently a (C₆-C₁₅)aryl (e.g., in some aspects, phenyl) optionally substituted with one or more substituents independently selected from halo, (C₁-C₃)alkoxy, (C₁-C₃)haloalkoxy, (C₁-C₃)alkyl or (C₁-C₃)haloalkyl.

In some aspects of the first embodiment, n is 1.

In some aspects of the first embodiment, n is 2.

In some aspects of the first embodiment, -L(R)ₙ is phenyl or benzyl.

Examples of amino acids of the first embodiment include the amino acids represented by the following structural formulas:

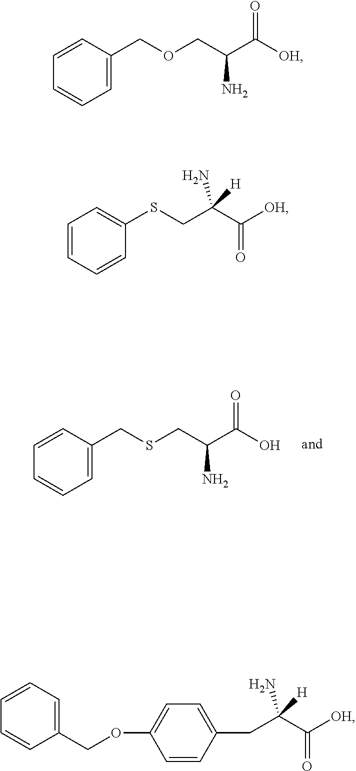 [SMILES: N[C@@H](COCc1ccccc1)C(=O)O ; N[C@@H](CSc1ccccc1)C(=O)O ; N[C@@H](CSCc1ccccc1)C(=O)O ; N[C@@H](Cc1ccc(OCc2ccccc2)cc1)C(=O)O]

or a pharmaceutically acceptable salt of any of the foregoing.

A second embodiment is an amino acid (e.g., in some aspects, a naturally-occurring amino acid and/or an L-amino acid), or a pharmaceutically acceptable salt thereof, wherein:
the amino acid is a hydroxyamino acid or thioamino acid;
the hydroxyl of the hydroxyamino acid is substituted with —PO₃H₂; and
the thiol of the thioamino acid is replaced with —SO₃H.

In some aspects of the second embodiment, the amino acid is not O-phospho-L-serine (OPLS), or a pharmaceutically acceptable salt thereof.

In some aspects of the second embodiment, the amino acid is threonine, cysteine, homocysteine or tyrosine.

Examples of amino acids of the second embodiment include L-homocysteic acid or O-phospho-L-tyrosine (OPLT), or a pharmaceutically acceptable salt of either of the foregoing.

A third embodiment is an amino acid (e.g., in some aspects, a naturally-occurring amino acid and/or an L-amino acid), or a pharmaceutically acceptable salt thereof, wherein:
(i) the amino acid is a hydroxyamino acid or thioamino acid; and
   the hydroxyl of the hydroxyamino acid or the thiol of the thioamino acid, respectively, is substituted with -L(R)ₙ;
   L is absent, or a (C₁-C₈)alkyl or (C₂-C₈)alkenyl optionally substituted with one or more fluoro;
   each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
   n is 1 or 2; or
(ii) the amino acid is tyrosine; and
   the hydroxyl of the tyrosine is substituted with (C₁-C₈)alkyl or (C₂-C₈)alkenyl optionally substituted with one or more fluoro; or
   the hydroxyl of the tyrosine is replaced with —H; or
(iii) the amino acid is a hydroxyamino acid or thioamino acid; and
   the hydroxyl of the hydroxyamino acid or the thiol of the thioamino acid, respectively, is substituted or replaced with —PO₃H₂, —SO₃H or —NO₂; or
(iv) the amino acid is a naturally-occurring hydroxyamino acid or naturally-occurring thioamino acid.

In some aspects of the third embodiment,
(i) the amino acid is a hydroxyamino acid or thioamino acid; and
   the hydroxyl of the hydroxyamino acid or the thiol of the thioamino acid, respectively, is substituted with -L(R)ₙ;
   L is absent, or a (C₁-C₈)alkyl or (C₂-C₈)alkenyl optionally substituted with one or more fluoro;
   each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
   n is 1 or 2; or
(ii) the amino acid is tyrosine; and
   the hydroxyl of the tyrosine is substituted with (C₁-C₈)alkyl or (C₂-C₈)alkenyl optionally substituted with one or more fluoro; or
   the hydroxyl of the tyrosine is replaced with -H.

In some aspects of the third embodiment, the amino acid is tyrosine; and the hydroxyl of the tyrosine is substituted with (C₁-C₈)alkyl or (C₂-C₈)alkenyl optionally substituted with one or more fluoro; or the hydroxyl of the tyrosine is replaced with —H. In some further aspects of these aspects, the hydroxyl of the tyrosine is substituted with (C₁-C₈)alkyl or (C₂-C₈)alkenyl optionally substituted with one or more fluoro. In yet further aspects of these aspects, the hydroxyl of the tyrosine is substituted with (C₁-C₈)alkyl optionally substituted with one or more fluoro.

In some aspects of the third embodiment:
the amino acid is a hydroxyamino acid or thioamino acid; and
   the hydroxyl of the hydroxyamino acid or the thiol of the thioamino acid, respectively, is substituted or replaced with —PO₃H₂, —SO₃H or —NO₂; or
(ii) the amino acid is a naturally-occurring hydroxyamino acid or naturally-occurring thioamino acid.

In some aspects of the third embodiment, the amino acid is a hydroxyamino acid or thioamino acid; and the hydroxyl of the hydroxyamino acid or the thiol of the thioamino acid, respectively, is substituted or replaced with —PO₃H₂, —SO₃H or —NO₂. In some further aspects of these aspects, the amino acid is serine, threonine, cysteine, homocysteine, tyrosine or hydroxyproline. In yet further aspects of these aspects, the amino acid is threonine, cysteine, homocysteine or tyrosine. In yet further aspects of these aspects, the amino acid is cysteine, homocysteine or tyrosine.

In some aspects of the third embodiment, the amino acid is a naturally-occurring hydroxyamino acid or thioamino acid. In some further aspects of these aspects, the amino acid is serine, threonine, cysteine, homocysteine, tyrosine or hydroxyproline. In yet further aspects of these aspects, the amino acid is tyrosine or hydroxyproline.

In some aspects of the third embodiment, the amino acid is not O-phospho-L-serine (OPLS), or a pharmaceutically acceptable salt thereof.

Alternative amino acids and/or alternative values for the variables in the amino acids of the third embodiment are as described in the first or second embodiment, or any aspect of the foregoing.

Examples of compounds of the disclosure include L-homocysteic acid, O-phospho-L-tyrosine (OPLT), L-methionine (e.g., L-methionine sulfate), 4-nitro-L-phenylalanine (e.g., 4-nitro-L-phenylalanine hydrate) as well as amino acids represented by the following structural formulas:

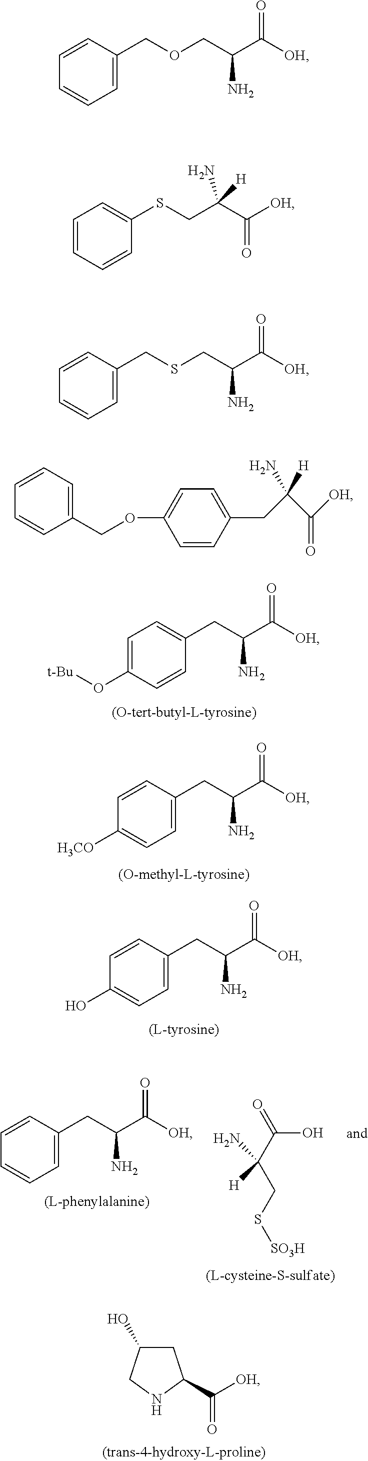

or a pharmaceutically acceptable salt of any of the foregoing. In some embodiments, an amino acid is selected from one of the following structural formulas:

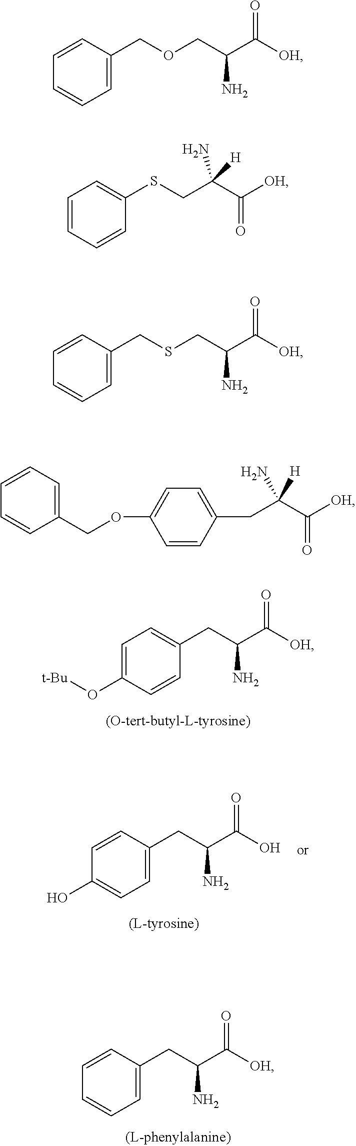

or a pharmaceutically acceptable salt of the foregoing. In some embodiments, an amino acid is selected from L-homocysteic acid, O-phospho-L-tyrosine (OPLT), L-methionine (e.g., L-methionine sulfate), 4-nitro-L-phenylalanine (e.g., 4-nitro-L-phenylalanine hydrate), L-cysteine-S-sulfate or trans-4-hydroxy-L-proline, or a pharmaceutically acceptable salt of the foregoing.

The compounds of the disclosure can be obtained from commercial sources or synthesized by a person of ordinary skill in the art using methods known in the art.

### Compositions and Kits

Typically, for administration to a subject, a compound of the disclosure is formulated with one or more pharmaceutically acceptable carriers. The disclosure provides such compositions, including pharmaceutical compositions. Thus, one embodiment is a composition (e.g., pharmaceutical composition) comprising a compound of the disclosure and a pharmaceutically acceptable carrier. The compositions described herein can be used in the methods described herein, e.g., to supply a compound of the disclosure.

Compounds and compositions described herein can also be in the form of formulations of lipid particles, such as liposomal formulations. Thus, one embodiment is a lipid particle (e.g., a liposome) comprising one or more lipids and a compound of the disclosure.

Also provided herein is a solid lipid particle (e.g., liposome) comprising at least one phospholipid (e.g., a phospholipid containing a C₄-C₃₀ acyl chain, such as a saturated C₄-C₃₀ acyl chain, as in dimyristoylphosphatidylcholine (DMPC)) and a therapeutic agent (e.g., a compound of the disclosure) that can embed in a lipid bilayer of the lipid particle. It has been found that oral administration of such solid lipid particles can be used to target the lipid particle (and thereby the therapeutic agent) to immune cells and/or lymph node(s), for example, and thereby enhance colocalization of the lipid particles and immune cells (e.g., in the lymph nodes) and/or enhance lymph node uptake of the lipid particles.

As used herein, “lipid particle” refers to a particle comprising at least one lipid, e.g., a phospholipid, such as a lysophospholipid. Examples of lipid particles include, liposomes, micelles and lipid nanoparticles. Lipid particles, such as liposomes, can be unilamellar or multilamellar. Lipid particles, such as liposomes, can have fluidic lipid membranes, or gel-like or solid lipid membranes, for example, lipid membranes that melt above normal body temperature of a human, or about 37° C. In some aspects, a lipid particle is a liposome. In some aspects, a lipid particle is a lipid nanoparticle. In some aspects, a lipid particle is solid. In some aspects, a lipid particle has a melting temperature above about 37° C., e.g., above about 40° C., above about 45° C., above about 50° C., above about 55° C. or about 55° C.

Examples of phospholipids include dimyristoylphosphatidylcholine (DMPC), 1,2-dioleoyl-sn-glycero-3-phosphocholine 18:1 Δ9-Cis PC (DOPC), 1,2-distearoyl-sn-glycero-3-phosphocholine 18:0 (DSPC), 1-palmitoyl-2-oleoyl-glycero-3-phosphocholine 16:0-18:1 (POPC), phosphatidylserine (PS), phosphatidylcholine (PC), phosphatidylethanolamine, phosphatidyl inositol, bisphosphatidyl glycerol, phosphatidic acid, phosphatidyl alcohol and phosphatidyl glycerol. Phospholipids can be saturated or unsaturated, i.e., contain one or more units of unsaturation, and can contain acyl chains of a variety of lengths. In some aspects, a phospholipid contains a C₄-C₃₀ acyl chain, e.g., a C₈-C₂₆, C₁₂-C₂₂, C₁₀-C₂₅, C₁₄-C₁₈ or C₁₆-C₂₆ acyl chain. Phospholipids can be obtained from various sources, both natural and synthetic. For example, PS can be obtained from porcine brain PS or plant-based soy (soya bean) PS. Egg PC and PS and synthetic PC are available commercially. In some aspects, a phospholipid is not PS, or a salt thereof (e.g., pharmaceutically acceptable salt thereof).

Typically, the molar percentage of a therapeutic agent (e.g., compound of the disclosure) in a lipid particle (e.g., liposome) comprising the therapeutic agent will be from about 1% to about 50%, e.g., from about 1% to about 35%, from about 1% to about 25%, from about 1% to about 15%, from about 3% to about 10%, from about 5% to about 50%, from about 5% to about 45%, from about 15% to about 40%, from about 25% to about 35%, about 1%, about 2%, about 3%, about 4%, about 5%, about 6%, about 7%, about 8%, about 9%, about 10%, about 15%, about 25%, about 30% or about 35%. In some aspects, the molar percentage of a therapeutic agent (e.g., compound of the disclosure) in a lipid particle (e.g., liposome) comprising the therapeutic agent will be less than 35%, e.g., less than 30%, less than 15%, or from about 1% to about 10%.

Typically, the molar percentage of lipid (taken individually or collectively) in a lipid particle (e.g., liposome) described herein will be from about 50% to about 99%, e.g., from about 50% to about 75%, from about 85% to about 99%, about 70%, about 75%, about 85%, about 90%, about 91%, about 92%, about 93%, about 94%, about 95%, about 96%, about 97%, about 98% or about 99%.

A compound of the disclosure can be encapsulated within a lipid particle, such as a liposome, described herein, bound (covalently or non-covalently) to a lipid head group or, preferably, embedded, in whole or in part, covalently or non-covalently, in a lipid bilayer (e.g., of a liposome). Without wishing to be bound by any particular theory, it is believed that compounds of the disclosure substituted with -L(R)ₙ may embed in a lipid bilayer of a liposome. Further, it is believed that the aryl and/or heteroaryl of R may embed so as to leave the amino acid residue of the compound of the disclosure exposed to the exterior of the liposome, thereby mimicking the natural surface presentation of, for example, PS. It is also believed that compounds of the disclosure wherein the amino acid is tyrosine wherein the hydroxyl of the tyrosine is substituted with (C₁-C₈)alkyl or (C₂-C₈)alkenyl optionally substituted with one or more fluoro, or the hydroxyl of the tyrosine is replaced with —H, and even tyrosine itself may embed in a lipid bilayer of a liposome. In preferred aspects of the lipid particles comprising a compound of the disclosure described herein, the compound of the disclosure is selected from those compounds identified herein as believed to embed in a lipid bilayer of a liposome.

In some aspects, the one or more lipids comprises a phospholipid, or a pharmaceutically acceptable salt thereof, e.g., 1,2-dimyristoyl-sn-glycero-3-phosphocholine (DMPC), or a pharmaceutically acceptable salt thereof. In some aspects, the phospholipid is a saturated phospholipid, e.g., a saturated phospholipid containing a C₄-C₃₀ acyl chain. In some aspects, the phospholipid is unsaturated, e.g., an unsaturated phospholipid containing a C₄-C₃₀ acyl chain. In some aspects, the phospholipid is selected from DMPC, DSPC, DOPC or POPC, or a pharmaceutically acceptable salt of the foregoing. In some aspects, the phospholipid is DMPC or DSPC, or a pharmaceutically acceptable salt of the foregoing.

In some aspects, a lipid particle (e.g., liposome) further comprises an antigen, such as any of the antigens described herein.

Lipid particles further comprising an antigen, and formulations comprising such lipid particles, are expected to be particularly useful for applications involving delivery of a gene therapy (e.g., a gene therapy comprising DNA and/or RNA) to a subject. The lipid particles are expected to promote co-presentation of the gene therapy and the compound of the disclosure to the immune system. Such particles can be formulated for oral and/or parenteral (e.g., subcutaneous, intramuscular, intravenous, intradermal) administration, e.g., as by injection.

Another embodiment is a composition (e.g., pharmaceutical composition) comprising a plurality of lipid particles (e.g., a plurality of lipid particles comprising a compound of the disclosure). In some aspects, a composition further comprises a pharmaceutically acceptable carrier.

Compositions described herein and, hence, compounds of the disclosure, may be administered orally, parenterally (including subcutaneously, intramuscularly, intravenously and intradermally), by inhalation spray, topically, rectally, nasally, buccally, vaginally or via an implanted reservoir. The terms “parenteral” and “parenterally,” as used herein, include subcutaneous, intracutaneous, intravenous, intramuscular, intraocular, intravitreal, intra-articular, intra-arterial, intra-synovial, intrasternal, intrathecal, intralesional, intrahepatic, intraperitoneal, intralesional and intracranial injection or infusion techniques. In some aspects, a composition described herein is administrable intravenously and/or intraperitoneally. In some aspects, a composition described herein is administrable orally. In some aspects, a composition described herein is administrable subcutaneously. Preferably, a composition described herein is administered orally, subcutaneously, intraperitoneally or intravenously.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some aspects, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the compound of the disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

A compound of the disclosure can also be in micro-encapsulated form with one or more excipients, as noted above. In such solid dosage forms, the compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another aspect, a compound of the disclosure can be provided in an extended (or “delayed” or “sustained”) release composition. This delayed-release composition comprises the compound of the disclosure and a delayed-release component. Such a composition allows targeted release of the compound, for example, into the lower gastrointestinal tract, for example, into the small intestine, the large intestine, the colon and/or the rectum. In certain aspects, a delayed-release composition further comprises an enteric or pH-dependent coating, such as cellulose acetate phthalates and other phthalates (e.g., polyvinyl acetate phthalate, methacrylates (Eudragits)). Alternatively, the delayed-release composition can provide controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intravenously. Compositions described herein for intravenous, subcutaneous, or intraperitoneal injection may contain an isotonic vehicle such as sodium chloride injection, Ringer's injection, dextrose injection, dextrose and sodium chloride injection, lactated Ringer's injection, or other vehicles known in the art.

Compositions described herein can also be administered in the form of suppositories for rectal administration. These can be prepared by mixing a compound of the disclosure with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For other topical applications, the compositions can be formulated in a suitable ointment containing the active component suspended or dissolved in one or more carriers. Carriers for topical administration of a compound described herein include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. Suitable carriers also include, but are not limited to, mineral oil, sorbitan monostearate, polysorbate 60, cetyl esters wax, cetearyl alcohol, 2-octyldodecanol, benzyl alcohol and water and penetration enhancers.

For ophthalmic use, compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents. Without wishing to be bound by any particular theory, it is believed that local delivery of a composition described herein, as can be achieved by nasal aerosol or inhalation, for example, can reduce the risk of systemic consequences of the composition, for example, consequences for red blood cells.

Other pharmaceutically acceptable carriers that can be used in the compositions described herein include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as a-, b-, and g-cyclodextrin, or chemically modified derivatives thereof, such as hydroxyalkylcyclodextrins, including hydroxylpropyl-b-cyclodextrins, such as 2- and/or 3-hydroxypropyl-b-cyclodextrins, or other solubilized derivatives thereof can also be advantageously used as a pharmaceutically acceptable carrier in the compositions described herein, e.g., to enhance delivery of agents described herein.

The compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

In some aspects, a composition described herein further includes one or more additional therapeutic agents, e.g., for use in combination with a compound of the disclosure. When a composition comprises a compound of the disclosure and one or more additional therapeutic agents, each agent should be present at a dosage level of between about 1% and about 100% and, more preferably, between about 5% and about 95% of the dosage normally administered in a monotherapy regimen.

Some embodiments provide a combination (e.g., pharmaceutical combination) comprising a compound of the disclosure (e.g., a composition described herein comprising a compound of the disclosure) and one or more additional therapeutic agents (e.g., one or more compositions comprising one or more additional therapeutic agents). Such combinations are particularly useful as, for example, when the compound of the disclosure and the one or more additional therapeutic agents are to be administered separately. In a combination provided herein, the compound of the disclosure and the one or more additional therapeutic agents can be administrable by the same route of administration or by different routes of administration.

One embodiment is a kit comprising a compound of the disclosure (e.g., a composition described herein comprising a compound of the disclosure) and an antigen (e.g., any of the antigens described herein, such as an antigenic therapy). In one aspect, the kit comprises a therapeutically effective amount of the compound of the disclosure (e.g., an amount sufficient to immunotolerize a subject to an antigen with which it is intended to be administered; a therapeutically effective amount of the compound to treat a disease, disorder or condition described herein). In some aspects, wherein the antigen is an antigenic therapy, the kit comprises a therapeutically effective amount of the antigenic therapy to treat the disease, disorder or condition. In some aspects, a kit further comprises an additional therapeutic agent(s) (e.g., a composition comprising an additional therapeutic agent(s)). In some aspects, the kit further comprises written instructions for administering the compound of the disclosure and/or the antigen and/or the additional agent(s) to a subject to treat a disease, disorder or condition described herein.

Suitable additional therapeutic agents include those described herein with respect to combination therapies.

The compositions described herein can be provided in unit dosage form. The amount of active ingredient that can be combined with a carrier to produce a unit dosage form will vary depending, for example, upon the subject being treated and the particular mode of administration. Typically, a unit dosage form will contain from about 1 to about 1000 mg of active ingredient(s), e.g., from about 1 to about 500 mg, from about 1 to about 250 mg, from about 1 to about 150 mg, from about 0.5 to about 100 mg, or from about 1 to about 50 mg of active ingredient(s).

In some aspects, the concentration of one or more therapeutic agents provided in a pharmaceutical composition is less than 100%, 90%, 80%, 70%, 60%, 50%, 40%, 30%, 20%, 19%, 18%, 17%, 16%, 15%,14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v; and/or greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some aspects, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v. In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods of Use

It has now been found that various compounds of the present disclosure and compositions described herein are capable of binding TIM, e.g., with higher affinity than its natural ligand, phosphatidyl serine, and diminishing the immune response.

One embodiment is a method of modulating the expression or activity of a T cell immunoglobulin and mucin domain (TIM) receptor, comprising contacting a cell (e.g., a cell expressing a TIM receptor, such as an immune cell) with a compound of the disclosure (e.g., a therapeutically effective amount of a compound of the disclosure). TIM receptors are type 1 cell-surface glycoproteins, and TIM1, TIM3 and TIM4, TIM receptors expressed in humans, have been identified as phosphatidylserine receptors. TIM1 is preferentially expressed on T-helper 2 cells, and operates as a potent costimulatory molecule for T-cell activation. TIM3 is preferentially expressed on T-helper 1 cells, type 1 T-cells and dendritic cells, and generates an inhibitory signal resulting in apoptosis of T-helper 1 cells and type 1 T-cells. TIM3 is also expressed on cytotoxic T-cells (e.g., CD8+), and generates an inhibitory signal resulting in apoptosis of cytotoxic T-cells (e.g., CD8+). TIM4 is expressed on antigen-presenting cells, and mediates phagocytosis of apoptotic cells, thereby promoting tolerance. In some aspects, the TIM receptor is a TIM3 receptor. In some aspects, the TIM receptor is a TIM4 receptor. In some aspects, the TIM receptor is a TIM1 receptor. “TIM” is also referred to, for example, in the literature, as “Tim”.

It is shown herein that agonists of TIM receptors inhibit the activity of at least toll-like receptor (TLR) 3 and TLR7, without substantially inhibiting the activity of TLRs 2 and 4, which largely recognize patterns presented by bacteria. Toll-like receptors (TLRs) form a family of pattern recognition receptors that are expressed on innate immune cells, and constitute the immune system's first line of defense against microbes. To date, ten human subtypes of TLRs have been identified. TLRs 1, 2, 4, 5, 6 and 10 are expressed on the cell surface, and TLRs 3, 7, 8 and 9 are localized to the endoplasmic reticulum, endosomes and lysosomes. TLRs 1, 2 and 6 recognize and bind to bacterial lipoproteins and glycolipids. TLRs 3, 7, 8 and 9 recognize and bind to nucleic acids, such as viral dsRNA (TLR3), ssRNA (TLR7, TLR8) and unmethylated CpG DNA (TLR9). TLR4 recognizes and binds to fibronectin and LPS. TLR5 recognizes and binds to bacterial flagellin. Without wishing to be bound by any particular theory, it is believed that compounds of the disclosure do not result in general immunosuppression, but may exert their effects in a more selective and specific manner.

Another embodiment is a method of modulating (e.g., inhibiting) the activity of TLR3, TLR7, TLR8 and/or TLR9, comprising contacting a cell (e.g., a cell expressing TLR3, TLR7, TLR8 and/or TLR9; an immune cell) with a compound of the disclosure (e.g., a therapeutically effective amount of a compound of the disclosure). In some aspects, the compound of the disclosure selectively modulates (e.g., inhibits) the activity of TLR3, TLR7, TLR8 and/or TLR9, e.g., modulates (e.g., inhibits) the activity of TLR3, TLR7, TLR8 and/or TLR9 to a greater extent than it modulates the activity of TLRs 1, 2, 4, 5, 6 and/or 10. For example, modulation (e.g., inhibition) of the activity of TLR3, TLR7, TLR8 and/or TLR9 by a compound of the disclosure can be more than two-fold greater, e.g., more than five-fold, more than 10-fold, more than 25-fold or more than 100-fold greater, than modulation (e.g., inhibition) of the activity of TLRs 1, 2, 4, 5, 6 and/or 10 by the compound. In some aspects, the compound does not modulate (e.g., inhibit) the activity of TLRs 1, 2, 4, 5, 6 and/or 10 to a measurable extent.

In some aspects of the methods described herein, the cell is an immune cell, e.g., a T-cell, such as a regulatory T-cell, a natural killer (NK) cell, a macrophage, a neutrophil, a myeloid-derived suppressor cell or a dendritic cell. In some aspects, an immune cell is FoxP3+ and/or CD4+, such as a FoxP3+ and/or CD4+ T-cell.

In some aspects of the methods described herein, the method is conducted in vitro. In other aspects of the methods described herein, the method is conducted in vivo. In some aspects, therefore, the cell (e.g., immune cell) is in a subject (e.g., a subject having a disease, disorder or condition described herein).

Another embodiment is a method of immunotolerizing a subject in need thereof to an antigen (e.g., an antigenic therapy), comprising administering to the subject a therapeutically effective amount of a compound of the disclosure, e.g., in the form of a composition described herein. Some aspects comprise administering to the subject the antigen, or an immunogenic fragment thereof, and a therapeutically effective amount of a compound of the disclosure, e.g., in the form of a composition described herein. Some aspects comprise administering to the subject a composition described herein comprising the compound of the disclosure and the antigen, or an immunogenic fragment thereof, e.g., a composition comprising a plurality of lipid particles, wherein each lipid particle comprises the compound of the disclosure and the antigen, or an immunogenic fragment thereof. In some aspects, the antigen, or an immunogenic fragment thereof, and the compound of the disclosure are administered to the subject in separate formulations.

Another embodiment is a method of immunotolerizing a subject in need thereof to an antigen, comprising administering to the subject the antigen and a therapeutically effective amount of a compound of the disclosure or composition described herein.

“Immunotolerizing,” as used herein, refers to diminishing and/or eliminating an immune response, e.g., to an antigen. An immune response can, for example, be evidenced by immunological hyperactivity, inflammatory cytokine release and/or activation of immune cells such as macrophages, neutrophils, eosinophils, T-cells and B-cells. “Immunotolerizing,” as used herein, contemplates, for example, decreasing immunological hyperactivity, inhibiting inflammatory cytokine release and/or inhibiting activation and/or neutralizing immune cells such as macrophages, neutrophils, eosinophils, T-cells and B-cells. In a clinical setting, immunotolerizing may be evidenced, for example, by reduced severity of autoimmune disease and/or improved activity of administered antigenic therapy.

Thus, the process of immunotolerizing can be viewed along a continuum that ranges from immunological hyperactivity to immunological hypoactivity to immunological non-responsiveness, e.g., to an antigen. “Immunotolerizing” contemplates incremental improvements along this continuum towards immunological non-responsiveness as well as inducing immunological hypoactivity or immunological non-responsiveness. In other words, immunotolerizing includes reducing the level of immune intolerance and inducing immune tolerance. In certain preferred embodiments described herein, the method induces immune tolerance.

In some aspects, a subject showing immune intolerance or an immune intolerant subject has a measurable immune response, e.g., to an antigen, such as measurable antibody production in response to an antigen. In some aspects, a subject showing immune tolerance or an immune tolerant subject, does not have a measurable immune response, e.g., to an antigen, such as measurable antibody production in response to an antigen. ELISA and/or activity assays, including those described herein, are known in the art, and can be used to measure antibody production indicative of immune intolerance.

In some autoimmune diseases, antibodies are not always present. Immune intolerance in such cases can be evident by clinical symptoms of autoimmune disease and/or the presence of self-reactive T-cells or B-cells and/or an increase in other inflammatory immune cells, such as neutrophils, eosinophils, etc. In some aspects, a subject showing immune intolerance or an immune intolerant subject (e.g., subject having an autoimmune disease, such as an autoimmune disease described herein) has a measurable cytokine response. For example, a subject having rheumatoid arthritis may have a measurable TNF-alpha response. In some aspects, a subject showing immune tolerance or an immune tolerant subject (e.g., subject having an autoimmune disease, such as an autoimmune disease described herein) does not have a measurable cytokine response.

Immunotolerizing can be achieved in a general or antigen-specific manner, resulting, for example, in general or antigen-specific immune tolerance, respectively. Indicators of general immunotolerization include, for example: (a) absence and/or diminishment of immunological hyperactivity and/or anti-inflammatory cytokine release; (b) neutralization of immune cells such as macrophages, neutrophils, eosinophils, T-cells and B-cells; and/or (c) an increase in number of regulatory T-cells and/or in the activity or level of tolerogenic T-cells (e.g., FoxP3+/CD4+0 cells). Indicators of antigen-specific immunotolerization include, for example: (a) an increase in the number of antigen-specific regulatory T-cells (e.g., FoxP3+/CD4+ cells); (b) a decrease in antigen-specific antibody titer and/or number of B cells, including antigen-specific memory B cells; (c) a decrease in IL-6 and/or IL-17; (d) an increase in TGF-beta, IL-10, IL-35, CD40, CD80 and/or CD86; and/or (e) hyporesponsiveness following re-challenge with an antigen. Techniques for evaluating these indicators are known in the art and described herein. For example, certain of the aforementioned indicators can be evaluated using culture conditions.

In autoimmune diseases, treatment with compounds of the disclosure leads to expansion of natural regulatory T-cells. Such treatment does not interfere with innate immune response, such as that mounted by innate immune cells responding to danger signals from pathogens, but results in general adaptive immunotolerization. Thus, immunotolerizing can be achieved herein without general innate immune suppression, such that, for example, a subject can still mount an innate immune response to an antigen (e.g., pathogen). In some aspects, immunotolerizing is general adaptive immunotolerization. In some aspects, immunotolerizing is antigen-specific, for example, resulting in reduced immune intolerance to a particular antigen(s) or immune tolerance to the particular antigen(s).

In some aspects, immunotolerizing is general, for example, resulting in generally reduced immune intolerance or general immune tolerance.

It will be understood that antigen-specific immunotolerizing can be achieved in accordance with the methods described herein not only by administering to a subject the specific antigen and a therapeutically effective amount of a compound of the disclosure or composition described herein, but also or alternatively by administering to a subject an immunogenic fragment of the specific antigen and a therapeutically effective amount of a compound of the disclosure or composition described herein.

As used herein, an “immunogenic fragment” of an antigen refers to a fragment of the antigen that induces an immune response to the antigen. An immunogenic fragment of an antigen may induce an immune response in a subject that is similar in extent to the immune response induced by the antigen itself, but need not induce the same extent of immune response as the antigen itself, so long as, when administered in accordance with the methods described herein, it has an immunotolerizing effect.

Another embodiment is a method of inhibiting or reducing an antigen-specific antibody titer in a subject, comprising administering to the subject a therapeutically effective amount of a compound of the disclosure, e.g., in the form of a composition described herein. Some aspects comprise administering to the subject the antigen, or an immunogenic fragment thereof, and a therapeutically effective amount of a compound of the disclosure, e.g., in the form of a composition described herein. Some aspects comprise administering to the subject a composition described herein comprising the compound of the disclosure and the antigen, or an immunogenic fragment thereof, e.g., a composition comprising a plurality of lipid particles, wherein each lipid particle comprises the compound of the disclosure and the antigen, or an immunogenic fragment thereof. In some aspects, the antigen, or an immunogenic fragment thereof, and the compound of the disclosure are administered to the subject in separate formulations.

Another embodiment is a method of inhibiting or reducing an antigen-specific antibody titer in a subject, comprising administering to the subject an antigen and a therapeutically effective amount of a compound of the disclosure or a composition described herein.

In some aspects of a method described herein, the antigen is an allergen, such as a food allergen or latex allergen. Examples of food allergens include peanut allergen, such as Ara h I or Ara h II; walnut allergen, such as Jug r I; brazil nut allergen, such as albumin; shrimp allergen, such as Pen a I; egg allergen, such as ovomucoid; milk allergen, such as bovine β-lactoglobin; wheat gluten antigen, such as gliadin); and fish allergen, such as parvalbumins. An example of a latex allergen is Hey b 7. Other allergens include antigen E, or Amb a I (ragweed pollen); protein antigens from grass, such as Lol p 1 (grass); dust mite allergens, such as, Der pI and Der PII (dust mites); Fel d I (domestic cat); and protein antigens from tree pollen, such as Bet v1 (white birch), and Cry j 1 and Cry j 2 (Japanese cedar). The allergen source listed in parentheses next to each allergen indicates the source with which the indicated allergen is typically associated.

Another embodiment is a method of inducing a population of regulatory T-cells in a subject, comprising administering to the subject a therapeutically effective amount of a compound of the disclosure, e.g., in the form of a composition described herein. Some aspects further comprise administering to the subject an antigen, or an immunogenic fragment thereof, in response to which the population of regulatory T-cells is being induced. Some aspects comprise administering to the subject a composition described herein comprising the compound of the disclosure and the antigen, or an immunogenic fragment thereof, e.g., a composition comprising a plurality of lipid particles, wherein each lipid particle comprises the compound of the disclosure and the antigen, or an immunogenic fragment thereof. In some aspects, the antigen, or an immunogenic fragment thereof, and the compound of the disclosure are administered to the subject in separate formulations.

Another embodiment is a method of inducing a population of regulatory T-cells in a subject, comprising administering to the subject a therapeutically effective amount of a compound of the disclosure or composition described herein.

Without wishing to be bound by any particular theory, it is believed that the compounds of the disclosure induce a population of regulatory T-cells primarily by expanding the population of natural regulatory T-cells (nT_{regs}, e.g., regulatory T-cells which are FoxP3+/NRP1+). The compounds of the disclosure also induce or upregulate inducible regulatory T-cells (iT_{regs}, e.g., FoxP3+ T-cells, FoxP3+/TIM3+ T-cells). Accordingly, in some aspects, a method of inducing a population of regulatory T-cells is a method of expanding a population of natural regulatory T-cells (e.g., regulatory T-cells which are FoxP3+/NRP1+), for example, without substantially inducing inducible regulatory T-cells. Neuropilin-1 (Nrp1) expression can be used to distinguish between natural and inducible regulatory T-cells, for example, as described herein. Thus, in some aspects, a method of inducing a population of regulatory T-cells is a method of inducing a population of regulatory T-cells expressing Nrp1 (e.g., FoxP3+/NRP1+ T-cells) as, for example, by expanding a population of natural regulatory T-cells. Without wishing to be bound by any particular theory, it is expected that the ability to expand a population of natural regulatory T-cells (e.g., regulatory T-cells which are FoxP3+/NRP1+), for example, without substantially inducing inducible regulatory T-cells, will be beneficial in treating autoimmune diseases without effecting general immunosuppression.

In some aspects, regulatory T-cells are FoxP3+, e.g., FoxP3+/TIM3+, FoxP3+/NRP1+. Whether a regulatory T-cell is positive (+) or negative (−) for any of the aforementioned markers can be determined, for example, by flow cytometry analysis.

Another embodiment is a method of increasing the activity or level of tolerogenic T-cells in a subject, comprising administering to the subject a therapeutically effective amount of a compound of the disclosure, e.g., in the form of a composition described herein.

Another embodiment is a method of increasing the activity or level of tolerogenic T-cells in a subject, comprising administering to the subject a therapeutically effective amount of a compound of the disclosure or composition described herein.

Another embodiment is a method of treating an autoimmune disorder in a subject, comprising administering to the subject a therapeutically effective amount of a compound of the disclosure or composition described herein. It will be appreciated that in autoimmune disorders, it may be desirable to induce general adaptive immunotolerization (e.g., immune tolerance) as, for example, by inducing a population of regulatory T-cells, or specific immunotolerization (e.g., immune tolerance) as, for example, by immunotolerizing a subject to a self-antigen associated with the autoimmune disorder, or an immunogenic fragment thereof. Thus, in some aspects of a method of treating an autoimmune disorder, the method further comprises administering (e.g., co-administering) a self-antigen associated with the autoimmune disorder, or an immunogenic fragment thereof, to the subject. Some aspects comprise administering to the subject a composition described herein comprising the compound of the disclosure and the self-antigen, or an immunogenic fragment thereof, e.g., a composition comprising a plurality of lipid particles, wherein each lipid particle comprises the compound of the disclosure and the self-antigen, or an immunogenic fragment thereof. In some aspects, the self-antigen, or an immunogenic fragment thereof, and the compound of the disclosure are administered to the subject in separate formulations. In some aspects of a method of treating an autoimmune disorder, the method further comprises co-administering a self-antigen associated with the autoimmune disorder to the subject.

Specific examples of autoimmune disorders treatable according to the methods described herein include achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II and III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenic purpura (TTP), thyroid eye disease (TED), Tolosa-Hunt syndrome (THS), transverse myelitis, Type 1 diabetes, ulcerative colitis (UC), undifferentiated connective tissue disease (UCTD), uveitis, vasculitis, vitiligo and Vogt-Koyanagi-Harada Disease.

In some aspects, the autoimmune disorder is a neurological autoimmune disorder. Examples of neurological autoimmune disorders include multiple sclerosis, neuromyelitis optica, myasthenia gravis, anti-myelin oligodendrocyte glycoprotein antibody disease (MOG), a MOG antibody-associated disorder (MOGAD, e.g., MOG-associated childhood demyelinating disease), autoimmune encephalitis, acute disseminated encephalomyelitis (ADEM), chronic meningitis, central nervous system vasculitis, Guillain-Barre syndrome, Hashimoto's thyroiditis, steroid responsive encephalopathy associated with autoimmune thyroiditis (SREAT), neurosarcoidosis, optic neuritis and transverse myelitis.

In some aspects, the autoimmune disorder is rheumatoid arthritis, systemic lupus erythematosus, inflammatory bowel disease, multiple sclerosis, type 1 diabetes mellitus, Guillain-Barre syndrome, chronic inflammatory demyelinating polyneuropathy, psoriasis, Graves' disease, Hashimoto's thyroiditis, myasthenia gravis or vasculitis.

In some aspects, the autoimmune disorder is multiple sclerosis. In some aspects, the multiple sclerosis is previously untreated. In alternative aspects, the multiple sclerosis is previously treated, e.g., with natalizumab (TYSABRI®) or glatiramer acetate. In some aspects, a compound of the disclosure is administered in combination with natalizumab and/or glatiramer acetate and, in some further aspects, the method further comprises administering natalizumab and/or glatiramer acetate to the subject.

Natalizumab is a recombinant humanized IgG4k monoclonal antibody produced in murine myeloma cells. Natalizumab binds to the a4-subunit of a4b1 and a4b7 integrins expressed on the surface of all leukocytes except neutrophils, and inhibits the a4-mediated adhesion of leukocytes to their counter-receptor(s). Natalizumab injection is indicated as monotherapy for the treatment of relapsing forms of multiple sclerosis, including clinically isolated syndrome, relapsing-remitting disease, and active secondary progressive disease, in adults. Glatiramer acetate injection is indicated for the treatment of relapsing forms of multiple sclerosis, including clinically isolated syndrome, relapsing-remitting disease, and active secondary progressive disease, in adults.

In some aspects, an autoimmune disorder is previously untreated. In alternative aspects, an autoimmune disorder is previously treated, e.g., with a standard of care therapy, such as natalizumab (TYSABRI®) or glatiramer acetate for multiple sclerosis.

Examples of self-antigens associated with autoimmune disorders include thyroid stimulating hormone receptor of the thyroid gland (Grave's disease); thyroid antigens, such as thyroid peroxidase (Hashimoto's thyroiditis); β cell antigens, such as glutamic acid decarboxylase and insulin (type I diabetes); cytochrome P450 antigens (Addison's disease); myelin proteins, such as myelin basic protein (multiple sclerosis); uveal antigens (uveitis); gastric parietal cell antigens, such as H⁺/ATPase and intrinsic factor (pernicious anemia); transglutaminase (gluten enteropathy); myocardial cell proteins, such as myosin (myocarditis, rheumatic heart disease); platelet antigens, such as GP IIb/IIIa (idiopathic thrombocytopenic purpura); red blood cell membrane proteins (autoimmune hemolytic anemia); neutrophil membrane proteins (autoimmune neutropenia); basement membrane antigens, such as type IV collagen .alpha.3 chain, (Goodpasture's disease); intrahepatic bile duct/mitochondrial antigens, such as 2-oxoacid dehydrogenase complexes (primary biliary cirrhosis); hepatocyte antigens, such as cytochrome P450 and 206 (autoimmune hepatitis); acetylcholine receptors (myasthenia gravis); desmogleins (pemphigus and other bullous diseases). The disorder listed in parentheses next to each self-antigen indicates the autoimmune disorder with which the indicated self-antigen is typically associated.

Compounds of the disclosure and compositions described herein are expected to be useful adjunctive therapies in the context of antigenic therapy, such as gene therapy, e.g., as by inhibiting an undesirable immune response to the antigenic therapy and/or enabling dosing and/or repeat dosing of the antigenic therapy. Another embodiment is a method of enhancing an antigenic therapy in a subject in need thereof (e.g., a subject receiving the antigenic therapy), comprising administering to the subject a compound of the disclosure or composition described herein. In some aspects, the compound of the disclosure or composition described herein is administered in an amount sufficient to immunotolerize the subject to the antigenic therapy. In some aspects, a therapeutically effective amount of the compound of the disclosure or composition described herein is administered. In some aspects, the method further comprises administering (e.g., co-administering) to the subject the antigenic therapy (e.g., a therapeutically effective amount of the antigenic therapy), for example, concurrently or sequentially with a compound of the disclosure or composition described herein. Some aspects comprise administering to the subject a composition described herein comprising the compound of the disclosure and the antigenic therapy, e.g., a composition comprising a plurality of lipid particles, wherein each lipid particle comprises the compound of the disclosure and the antigenic therapy. In some aspects, the antigenic therapy and the compound of the disclosure are administered to the subject in separate formulations.

Another embodiment is a method of treating a disease, disorder or condition in a subject in need thereof (e.g., a subject receiving an antigenic therapy), comprising administering to the subject a compound of the disclosure or composition described herein. In some aspects, the compound of the disclosure or composition described herein is administered in an amount sufficient to immunotolerize the subject to an antigenic therapy. In some aspects, the method further comprises administering (e.g., co-administering) to the subject the antigenic therapy (e.g., a therapeutically effective amount of the antigenic therapy), for example, concurrently or sequentially with a compound of the disclosure or composition described herein. Some aspects comprise administering to the subject a composition described herein comprising the compound of the disclosure and the antigenic therapy, e.g., a composition comprising a plurality of lipid particles, wherein each lipid particle comprises the compound of the disclosure and the antigenic therapy. In some aspects, the antigenic therapy and the compound of the disclosure are administered to the subject in separate formulations.

In some aspects, the antigenic therapy is an antibody therapy (e.g., monoclonal antibody therapy), including chimeric, humanized and fully-human antibody therapies. Specific examples of antibody therapies include anti-tumor necrosis factor (anti-TNF) therapies, such as adalimumab (Humira®; for rheumatoid arthritis, juvenile idiopathic arthritis, psoriatic arthritis, ankylosing spondylitis, Crohn's disease, ulcerative colitis, plaque psoriasis, hidradenitis suppurativa, uveitis) and infliximab (Remicade®, for Crohn's disease, pediatric Crohn's disease, ulcerative colitis, pediatric ulcerative colitis, rheumatoid arthritis, ankylosing spondylitis, psoriatic arthritis, plaque psoriasis), golimumab (Simponi®, for rheumatoid arthritis, psoriatic arthritis, ankylosing spondylitis, polyarticular juvenile idiopathic arthritis), etanercept (Enbrel®, for rheumatoid arthritis, polyarticular juvenile idiopathic arthritis, psoriatic arthritis, ankylosing spondylitis, plaque psoriasis) and certolizumab pegol (Cimzia®, for Crohn's disease, rheumatoid arthritis, psoriatic arthritis, ankylosing spondylitis, non-radiographic axial spondyloarthritis, plaque psoriasis).

In some aspects, the antigenic therapy is a protein replacement therapy, for example, enzyme replacement therapy. Examples of protein replacement therapies include replacement therapies for coagulation disorders, such as Factor VIII and Factor IX for hemophilia A and B; enzyme replacement therapies for lysosomal storage diseases, such as alglucosidase alfa (Myozyme® and Lumizyme®) for Pompe disease; alpha-L-iduronidase for Hurler syndrome; and adenosine deaminase for adult-type adenosine deaminase deficiency.

In some aspects, the antigenic therapy is a gene therapy. Gene therapies typically work by one of the following three mechanisms: (1) by supplying a subject with a healthy copy of a disease-causing gene (as does voretigene neparvovec-rzyl (Luxturna®), for example); (2) by inactivating a disease-causing gene (as may ASOs and siRNA, for example); or (3) by introducing a gene into the body to help treat a disease. Gene therapies include DNA (e.g., antisense oligonucleotides (ASOs)) and RNA (e.g., siRNA), which can be delivered to a subject in vivo or ex vivo via a variety of products. In vivo gene delivery products include plasmid DNA, viral vectors (e.g., AAV, such as AAV9) and non-viral vectors, such as bacterial vectors or lipid nanoparticles. Ex vivo gene delivery products include subject-derived cellular gene therapy products. Gene therapies also include gene editing technologies, such as CRISPR. Specific examples of gene therapies include voretigene neparvovec-rzyl (Luxturna®, for retinal dystrophy); and onasemnogene abeparvovec-xioi (Zolgensma®, for pediatric spinal muscular atrophy).

In some aspects, the antigenic therapy is a cellular therapy. An example of a cellular therapy is axicabtagene ciloleucel (Yescarta®, for relapsed or refractory large B-cell lymphoma). Another example of a cellular therapy is CAR-T cells.

Alloantigens, antigens present in some but not all individuals of a species and recognized as foreign by those that lack it, are often the basis for graft rejection reactions. Accordingly, another embodiment is a method of treating graft-versus-host disease in a subject in need thereof, comprising administering to the subject a therapeutically effective amount of compound of the disclosure or composition described herein.

Examples of alloantigens include, but are not limited to, major histocompatability complex (MHC) class I and class II antigens, minor histocompatability antigens, endothelial glycoproteins, such as blood group antigens, and carbohydrate determinants.

Another embodiment is a method for promoting wound healing in a subject in need thereof, comprising administering to the subject a therapeutically effective amount of compound of the disclosure or composition described herein.

In some aspects of any of the methods described herein, particularly those involving compositions that do not comprise an antigen, wherein antigen-specific and/or antigen-exclusive immune tolerance is desired (e.g., when the composition is being administered to immunotolerize a subject to an antigenic therapy), the method further comprises administering an antigen, or an immunogenic fragment thereof (e.g., an antigen), to the subject. When an antigen, or an immunogenic fragment thereof, is administered herein in order to immunotolerize a subject to an antigen in an antigen-specific and/or antigen-exclusive manner, the antigen, or an immunogenic fragment thereof, and the compound of the disclosure or composition described herein are preferably co-administered. Thus, in some aspects, the antigen, or an immunogenic fragment thereof (e.g., the antigen), and the compound of the disclosure or composition described herein are co-administered.

As used herein, “co-administer,” “co-administration” and the like refer to simultaneous or nearly simultaneous but sequential administration of two or more agents (e.g., a compound of the disclosure and an antigen) via the same route of administration at the same or nearly the same site on the body of a subject.

When co-administration is simultaneous (e.g., concurrent), a first agent (e.g., a compound of the disclosure) and a second agent (e.g., an additional therapeutic agent, an antigen, or an immunogenic fragment thereof) can be in separate formulations or the same formulation. Alternatively, the first and second agents can be administered sequentially as separate compositions. When co-administration is sequential, administration of subsequent composition(s) occurs within 24 hours of administration of a first composition and, preferably, within 12 hours, for example, within 10 hours, 5 hours, 4 hours, 3 hours, 2 hours, 60 minutes, 30 minutes, 15 minutes, 10 minutes or 5 minutes, of administration of the first composition. Typically, when co-administration is sequential, the administration of subsequent composition(s) follows immediately after completion of administration of the first composition, taking into account any manipulations that a clinician or subject administering the compositions may need to engage in to ready subsequent composition(s) for administration.

When co-administration is oral, the site of administration is the mouth, and the two or more agents being co-administered are administered at the same site, by mouth, whether or not they are given in a single formulation or separate formulations. When co-administration is by injection of two or more compositions, however, the site of administration is more typically nearly the same. In such situations, the anatomical sites of administration are typically less than 2 inches apart from one another, for example, less than about 0.5 inches, less than about 1 inch or less than about 1.5 inches from one another.

In some aspects, an antigen and a compound of the disclosure or composition described herein are co-administered, and administration of the antigen precedes administration of the compound of the disclosure or composition described herein. In alternative aspects, an antigen and a compound of the disclosure or composition described herein are co-administered, and administration of the compound of the disclosure or composition described herein precedes administration of the antigen.

Co-administration can occur by any route of administration described herein. In some aspects, a compound of the disclosure or composition described herein and an antigen are co-administered orally. In some aspects, a compound of the disclosure or composition described herein and an antigen are co-administered subcutaneously.

Without wishing to be bound by any particular theory, it is believed that it is desirable for a subject's immune system to encounter antigen and compound of the disclosure together, or for the antigen and compound of the disclosure to be “co-presented” to a subject's immune system. When a compound of the disclosure is co-administered with an antigen and the antigen is a protein, such as a protein replacement therapy, co-administration, as, for example, by injection of separate formulations of antigen and compound of the disclosure, is expected to provide for effective co-presentation of the compound of the disclosure and the antigen to a subject's immune system. In such applications, the compound of the disclosure may, but need not be, incorporated into a lipid particle. In preferred aspects of such applications, co-administration is subcutaneous, e.g., by injection. In applications involving delivery of a gene therapy (e.g., a gene therapy comprising DNA and/or RNA), it may be desirable, in order to promote effective co-presentation of the gene therapy and the compound of the disclosure to a subject's immune system, to formulate the gene therapy and the compound of the disclosure into lipid particles comprising the gene therapy and the compound of the disclosure. In preferred aspects, such particles are formulated for oral and/or parenteral (e.g., subcutaneous, intramuscular, intravenous, intradermal) administration, e.g., as by injection.

Without wishing to be bound by any particular theory, it is believed that amino acids wherein the amino acid is a hydroxyamino acid or thioamino acid, and the hydroxyl of the hydroxyamino acid or the thiol of the thioamino acid, respectively, is substituted or replaced with —PO₃H₂, —SO₃H or —NO₂, and amino acids wherein the amino acid is a naturally-occurring hydroxyamino acid or thioamino acid, such as hydroxyproline, have physical characteristics, such as enhanced water solubility, that promote their formulation without a lipid particle. Use of such compounds, e.g., in accordance with the methods disclosed herein, may be advantageous in aspects wherein effective co-presentation of the compound of the disclosure and the antigen to a subject's immune system can be achieved in the absence of a lipid particle, e.g., by co-administration of separate formulations of antigen and compound of the disclosure.

Also without wishing to be bound by any particular theory, it is believed that particular compounds of the disclosure identified herein embed in a liposome. Use of such compounds, e.g., in accordance with the methods disclosed herein, may be advantageous in aspects wherein effective co-presentation of the compound of the disclosure and the antigen to the subject's immune system is promoted by incorporation of compound of the disclosure and antigen into a lipid particle comprising the compound of the disclosure and the antigen.

A compound of the disclosure or a composition described herein can also be administered in combination with one or more non-antigenic therapies to treat a disease, disorder or condition. When administered “in combination” with such non-antigenic therapies, the compound of the disclosure or composition described herein can be administered before, after or concurrently with the other therapy(ies) (e.g., additional therapeutic agent(s)). When administered simultaneously (e.g., concurrently), the compound of the disclosure and another therapeutic agent can be in separate formulations or the same formulation. Alternatively, the compound of the disclosure and another therapeutic agent can be administered sequentially, either at approximately the same time or at different times, as separate compositions. When the compound of the disclosure and the other therapy (e.g., therapeutic agent) are administered as separate formulations or compositions, the compound of the disclosure and the other therapy can be administered by the same route of administration or by different routes of administration. A skilled clinician can determine appropriate timing for administration of each therapy being used in combination (e.g., timing sufficient to allow an overlap of the pharmaceutical effects of the therapies). Typically, a combination therapy will provide beneficial effects of the drug combination in treating the diseases, conditions or disorders described herein.

In some aspects, a method described herein further comprises administering to the subject (e.g., a therapeutically effective amount of) an additional, non-antigenic therapy(ies), e.g., in combination with a compound of the disclosure or composition described herein. In some aspects, the compound of the disclosure or composition described herein is administered before the additional therapy(ies). In some aspects, the compound of the disclosure or composition described herein is administered after the additional therapy(ies). In some aspects, the compound of the disclosure or composition described herein is administered concurrently with the additional therapy(ies).

A therapeutically effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

A compound of the disclosure, composition described herein, antigen or other therapeutic agent can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound, antigen and/or therapeutic agent, respectively, and the particular disease to be treated. Administration can be local or systemic as indicated. The preferred mode of administration can vary depending on the particular compound or agent.

In some aspects, administration (e.g., of a compound of the disclosure or composition described herein and/or an antigen) is oral. In some aspects, administration (e.g., of a compound of the disclosure or composition described herein and/or an antigen) is intravenous. In some aspects, administration (e.g., of a compound of the disclosure or composition described herein and/or an antigen) is subcutaneous.

A compound of the disclosure or composition described herein can be administered, in accordance with the methods disclosed herein, prophylactically, as when a subject with no known immune intolerance to an antigenic therapy is co-administered a compound of the disclosure or composition described herein with the antigenic therapy. A compound of the disclosure or composition described herein can also or alternatively be administered, in accordance with the methods disclosed herein, therapeutically, as when a subject has demonstrated immune intolerance to an antigen (e.g., an allergic reaction, graft rejection). Accordingly, in some aspects, a subject has no known immune intolerance to an antigen, for example, because the subject is naïve to the antigen. In some aspects, a subject has no known immune intolerance to an antigen after having been administered and/or exposed to the antigen. In some aspects, a subject is immune intolerant to an antigen, for example, developed immune intolerance after having been administered and/or exposed to the antigen or is inherently immune intolerant to the antigen.

A compound of the disclosure or composition described herein can be administered (e.g., co-administered), in accordance with the methods disclosed herein, upon a first exposure to an antigen, as when a compound of the disclosure or composition described herein is administered with a first dose of an antigenic therapy. Also or alternatively, a compound of the disclosure or composition described herein can be administered (e.g., co-administered), in accordance with the methods disclosed herein, upon a second or further additional exposure to an antigen, as when a compound of the disclosure or composition described herein is administered with a second or further additional dose (e.g., a repeat dose) of an antigenic therapy.

The methods described herein are intended to reduce immune intolerance to an antigen for an extended period of time, for example, a period of time necessary to treat a disease, disorder or condition with an antigenic therapy described herein, for life of a subject. Accordingly, in some aspects of the methods described herein, the method further comprises administering to the subject the antigen (e.g., antigenic therapy, such as a therapeutically effective amount of the antigenic therapy) to the subject in the absence of the compound of the disclosure or composition described herein.

However, a subject's immune intolerance may increase over time following a method described herein, e.g., following subsequent exposure(s) to the antigen. In such cases, the methods described herein can be repeated, for example, as a “booster” vaccine is repeated, to re-immunotolerize the subject to the antigen.

A compound of the disclosure or other therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. In some embodiments, administration (e.g., of a compound of the disclosure) is oral. In some embodiments, administration (e.g., of a compound of the disclosure) is intravenous. The preferred mode of administration can vary depending on the particular compound or agent. Typically, a compound of the disclosure or other therapeutic agent will be administered from about 1 to about 6 (e.g., 1, 2, 3, 4, 5 or 6) times per day, also or alternatively, as an infusion (e.g., a continuous infusion).

It has been shown that orally administered liposomes, such as those described herein, can reach the lymph node, and colocalize with immune cells, including B-cell and T-cells, in the lymph node. Accordingly, also provided herein is a method of delivering a therapeutic agent (e.g., a compound of the disclosure) to a lymph node of a subject (e.g., a subject in need thereof), comprising orally administering to the subject a therapeutically effective amount of a composition comprising a plurality of lipid particles (e.g., solid lipid particles), wherein each lipid particle comprises at least one phospholipid (e.g., a phospholipid containing a C₄-C₃₀ acyl chain, such as a saturated C₄-C₃₀ acyl chain, as in dimyristoylphosphatidylcholine (DMPC)) and a therapeutic agent that can embed in a lipid bilayer of the lipid particle (e.g., a compound of the disclosure).

A compound of the disclosure or other therapeutic agent can be administered in a dosage ranging from about 0.001 mg/kg to about 100 mg/kg of body weight or, alternatively, in a dosage ranging from about 1 mg/dose to about 5,000 mg/dose, every 4 to 120 hours, or according to the requirements of the particular agent. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Suitable dosages can be from about 1 mg/dose to about 5,000 mg/dose, from about 10 mg/dose to about 2,500 mg/dose or from about 100 mg/dose to about 1,000 mg/dose.

Doses lower or higher than those recited above may be required. Specific dosage and treatment regimens for any particular patient will depend, for example, upon a variety of factors, such as the activity of the specific agent employed, the age, body weight, general health status, sex, diet, time of administration, rate of excretion, drug combination, the severity and course of the disease, condition or symptoms, the subject's disposition to the disease, condition or symptoms, and the judgment of the treating physician. Determining the dosage for a particular agent, subject and disease, disorder or condition is within the abilities of one of skill in the art.

### NUMBERED EMBODIMENTS

1. A lipid particle comprising one or more lipids, or a pharmaceutically acceptable salt thereof, and an amino acid, or a pharmaceutically acceptable salt thereof, wherein: the amino acid is a hydroxyamino acid or thioamino acid;
   the hydroxyl of the hydroxyamino acid or the thiol of the thioamino acid, respectively, is substituted with -L(R)ₙ;
   L is absent, or a (C₁-C₈)alkyl or (C₂-C₈)alkenyl optionally substituted with one or more fluoro;
   each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2.
2. The lipid particle of embodiment 1, in the form of a liposome.
3. The lipid particle of embodiment 1 or 2, wherein the amino acid is a naturally-occurring amino acid.
4. The lipid particle of any one of embodiments 1-3, wherein the amino acid is serine, threonine, cysteine, homocysteine or tyrosine.
5. The lipid particle of any one of embodiments 1-4, wherein the amino acid is a L-amino acid.
6. The lipid particle of any one of embodiments 1-5, wherein L is absent or (C₁-C₃)alkyl optionally substituted with one or more fluoro.
7. The lipid particle of any one of embodiments 1-6, wherein each R is independently a (C₆-C₁₅)aryl optionally substituted with one or more substituents independently selected from halo, (C₁-C₃)alkoxy, (C₁-C₃)haloalkoxy, (C₁-C₃)alkyl or (C₁-C₃)haloalkyl.
8. The lipid particle of any one of embodiments 1-7, wherein n is 1.
9. The lipid particle of any one of embodiments 1-8, wherein -L(R)ₙ is phenyl or benzyl.
10. The lipid particle of any one of embodiments 1-9, wherein the one or more lipids includes a phospholipid, or a pharmaceutically acceptable salt thereof.
11. The lipid particle of any one of embodiments 1-10, wherein the one or more lipids includes 1,2-dimyristoyl-sn-glycero-3-phosphocholine (DMPC), or a pharmaceutically acceptable salt thereof.
12. The lipid particle of any one of embodiments 1-11, wherein the amino acid is represented by one of the following structural formulas:

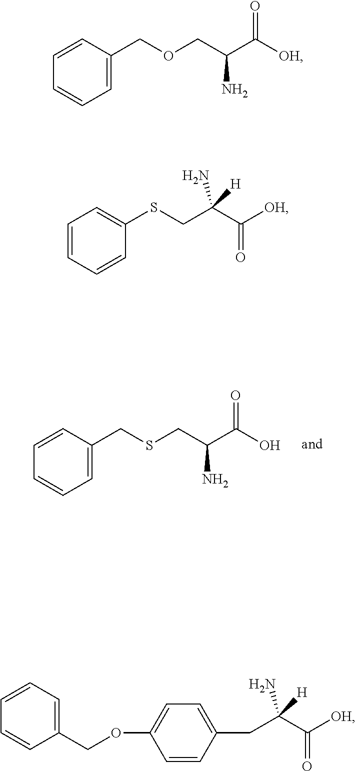 [SMILES: N[C@@H](COCc1ccccc1)C(=O)O ; N[C@@H](CSc1ccccc1)C(=O)O ; N[C@@H](CSCc1ccccc1)C(=O)O ; N[C@@H](Cc1ccc(OCc2ccccc2)cc1)C(=O)O]

or a pharmaceutically acceptable salt of any of the foregoing.
13. The lipid particle of any one of embodiments 1-12, further comprising an antigen.
14. A composition comprising a plurality of lipid particles according to any one of embodiments 1-12.
15. The composition of embodiment 14, further comprising a pharmaceutically acceptable carrier.
16. The composition of embodiment 14 or 15, further comprising an antigen.
17. A composition comprising a plurality of lipid particles according to embodiment 13.
18. The composition of embodiment 17, further comprising a pharmaceutically acceptable carrier.
19. A composition comprising an antigen and an amino acid, or a pharmaceutically acceptable salt thereof, wherein:
   the amino acid is a hydroxyamino acid or thioamino acid;
   the hydroxyl of the hydroxyamino acid is substituted with —PO₃H₂; and
   the thiol of the thioamino acid is replaced with —SO₃H,
   provided the amino acid is not O-phospho-L-serine.
20. A kit comprising a composition of embodiment 14 or 15 and an antigen.
21. A method of immunotolerizing a subject in need thereof to an antigen, comprising administering to the subject the antigen and a therapeutically effective amount of:
   (i) a composition of embodiment 14 or 15; or
   (ii) a composition comprising an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
22. A method of inducing a population of regulatory T-cells in a subject, comprising administering to the subject a therapeutically effective amount of:
   (i) a composition of embodiment 14 or 15; or
   (ii) a composition comprising an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
23. A method of inhibiting or reducing an antigen-specific antibody titer in a subject, comprising administering to the subject an antigen and a therapeutically effective amount of:
   (i) a composition of embodiment 14 or 15; or
   (ii) a composition comprising an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
24. A method of increasing the activity or level of tolerogenic T-cells in a subject, comprising administering to the subject a therapeutically effective amount of:
   (i) a composition of embodiment 14 or 15; or
   (ii) a composition comprising an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
25. The method of embodiment 22 or 24, further comprising administering an antigen to the subject.
26. The method of any one of embodiments 21-25, wherein the antigen and the composition are co-administered.
27. A method of immunotolerizing a subject in need thereof to an antigen, comprising administering to the subject a therapeutically effective amount of:
   (i) a composition comprising a plurality of lipid particles according to any one of embodiments 1-12 and the antigen;
   (ii) a composition comprising a plurality of lipid particles according to any one of embodiments 1-12, wherein the lipid particles further comprise the antigen; or
   (iii) a composition comprising the antigen and an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
28. A method of inducing a population of regulatory T-cells in a subject in response to an antigen, comprising administering to the subject a therapeutically effective amount of:
   (i) a composition comprising a plurality of lipid particles according to any one of embodiments 1-12 and the antigen;
   (ii) a composition comprising a plurality of lipid particles according to any one of embodiments 1-12, wherein the lipid particles further comprise the antigen; or
   (iii) a composition comprising the antigen and an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
29. A method of inhibiting or reducing an antigen-specific antibody titer in a subject, comprising administering to the subject a therapeutically effective amount of:
   (i) a composition comprising a plurality of lipid particles according to any one of embodiments 1-12 and the antigen;
   (ii) a composition comprising a plurality of lipid particles according to any one of embodiments 1-12, wherein the lipid particles further comprise the antigen; or
   (iii) a composition comprising the antigen and an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
30. A method of increasing the activity or level of tolerogenic T-cells in a subject, comprising administering to the subject a therapeutically effective amount of:
   (i) a composition comprising a plurality of lipid particles according to any one of embodiments 1-12 and an antigen;
   (ii) a composition comprising a plurality of lipid particles according to any one of embodiments 1-12, wherein the lipid particles further comprise an antigen; or
   (iii) a composition comprising an antigen and an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
31. The lipid particle of embodiment 13, the composition of any one of embodiments 16-19, the kit of embodiments 20 or the method of any one of embodiments 21-30, wherein the antigen is a protein.
32. The lipid particle of embodiment 13, composition of any one of embodiments 16-19, kit of embodiment 20 or method of any one of embodiments 21-31, wherein the antigen is a self-antigen.
33. The lipid particle of embodiment 13, composition of any one of embodiments 16-19, kit of embodiment 20 or method of any one of embodiments 21-31, wherein the antigen is a foreign antigen.
34. The lipid particle, composition, kit or method of embodiment 33, wherein the foreign antigen is an antigenic therapy.
35. The lipid particle, composition, kit or method of embodiment 33 or 34, wherein the foreign antigen is a therapeutic protein.
36. The lipid particle, composition, kit or method of any one of embodiments 33-35, wherein the foreign antigen is an enzyme replacement therapy.
37. The lipid particle, composition, kit or method of embodiment 33 or 34, wherein the foreign antigen is a cellular or gene therapy.
38. A method of treating an autoimmune disorder in a subject in need thereof, comprising administering to the subject a therapeutically effective amount of:
   (i) a composition of embodiment 14 or 15; or
   (ii) a composition comprising an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
39. The method of embodiment 38, further comprising administering a self-antigen associated with the autoimmune disorder to the subject.
40. A method of treating an autoimmune disorder in a subject in need thereof, comprising administering to the subject a therapeutically effective amount of:
   (i) a composition comprising a plurality of lipid particles according to any one of embodiments 1-12 and a self-antigen associated with the autoimmune disorder;
   (ii) a composition comprising a plurality of lipid particles according to any one of embodiments 1-12, wherein the lipid particles further comprise a self-antigen associated with the autoimmune disorder; or
   (iii) a composition comprising a self-antigen associated with the autoimmune disorder and an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
41. The method of any one of embodiments 38-40, wherein the autoimmune disorder is rheumatoid arthritis, systemic lupus erythematosus, inflammatory bowel disease, multiple sclerosis, type 1 diabetes mellitus, Guillain-Barre syndrome, chronic inflammatory demyelinating polyneuropathy, psoriasis, Graves' disease, Hashimoto's thyroiditis, myasthenia gravis or vasculitis.
42. A method of treating a disease, disorder or condition in a subject in need thereof with an antigenic therapy, comprising administering to the subject the antigenic therapy and, in an amount sufficient to immunotolerize the subject to the antigenic therapy:
   (i) a composition of embodiment 14 or 15; or
   (ii) a composition comprising an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
43. A method of treating a disease, disorder or condition in a subject in need thereof with an antigenic therapy, comprising administering to the subject in an amount sufficient to immunotolerize the subject to the antigenic therapy:
   (i) a composition comprising a plurality of lipid particles according to any one of embodiments 1-12 and the antigenic therapy;
   (ii) a composition comprising a plurality of lipid particles according to any one of embodiments 1-12, wherein the lipid particles further comprise the antigenic therapy; or
   (iii) a composition comprising the antigenic therapy and an amino acid, or a pharmaceutically acceptable salt thereof, wherein:
      the amino acid is a hydroxyamino acid or thioamino acid;
      the hydroxyl of the hydroxyamino acid is substituted with —PO₃H₂; and
      the thiol of the thioamino acid is replaced with —SO₃H,
      provided the amino acid is not O-phospho-L-serine.
44. The method of embodiment 39, 41 or 42, wherein the self-antigen or the antigenic therapy and the composition are co-administered.
45. The method of any one of embodiments 42-44, further comprising administering to the subject a therapeutically effective amount of the antigenic therapy in the absence of the composition.
46. The method of any one of embodiments 42-45, wherein the antigenic therapy is a gene therapy or an enzyme replacement therapy.
47. The method of any one of embodiments 38-46, further comprising administering to the subject an additional therapeutic agent.
48. The method of any one of embodiments 21-47, wherein the composition is administered orally.
49. The method of any one of embodiments 21-47, wherein the composition is administered subcutaneously.
50. The method of any one of embodiments 21-49, wherein the composition comprises an amino acid, or a pharmaceutically acceptable salt thereof, wherein the amino acid is a hydroxyamino acid or thioamino acid, provided the amino acid is not serine; the hydroxyl of the hydroxyamino acid is substituted with —PO₃H₂; and the thiol of the thioamino acid is replaced with —SO₃H.
51. The composition of embodiment 19 or method of embodiment 50, wherein the amino acid is a naturally-occurring amino acid.
52. The composition or method of embodiment 51, wherein the amino acid is threonine, cysteine, homocysteine or tyrosine.
53. The composition or method of embodiment 51 or 52, wherein the amino acid is a L-amino acid.
54. The composition or method of any one of embodiments 50-53, wherein the amino acid is L-homocysteic acid or O-phospho-L-tyrosine, or a pharmaceutically acceptable salt of any of the foregoing.

### EXEMPLIFICATION

### Example 1

### Reducing Immune Response to FVIII in B57BL6 Mice Using O-phospho-L-tyrosine (OPLT)

TIM receptors have been identified as phosphatidylserine (PS)-binding receptors. TIM receptors mediate the phagocytosis of apoptotic cells and play a role in maintaining tolerance towards self. TIM3 is expressed on the surface of naïve T-cells, and can recognize and bind PS, inducing T-cell signaling arrest. This is believed to play an important role in maintaining tolerance towards self

O-Phospho-L-serine (OPLS) is the head group of PS, TIM's natural ligand. Through in vitro screening, several compounds were identified that can bind TIM with higher affinity than PS. In this study, OPLT was evaluated for its tolerogenic effects. Based on this analysis, OPLT is expected to be more potent than OPLS.

In this study, FVIII was used as a model antigen. FVIII is used in the treatment of hemophilia A. It is reported that about 30% of patients treated with FVIII will develop anti-FVIII antibodies. Those antibodies can neutralize FVIII effects, rendering therapy ineffective. It is desirable to develop an approach to mitigate the immunogenicity of enzyme replacement therapy, such as FVIII.

All animal studies were conducted in compliance with Tufts University/Tufts Medical Center & Human Nutrition Research Center on Aging Animals were housed four in a cage, and had access to food and water ad libitum.

OPLT was dissolved in sterile PBS, and pH was adjusted to approximately 7.5. OPLS, a comparator compound, was dissolved in sterile PBS, and pH was adjusted to approximately 7.5. FVIII in PBS was used as a control. For treatment, FVIII was co-mixed with OPLT, OPLS or diluted in PBS before administration, as per group assignment for each animal. For re-challenge, FVIII was administered alone SC to all animals regardless of group assignment. Based on published reports, OPLS showed efficacy at 5 mg/injection SC. For a head-to-head comparison, 5 mg/injection was selected for OPLT. FVIII was dosed at 1 μg/injection.

The study was conducted in two parts: a treatment phase and a re-challenge phase. The objective of the treatment phase was to test the ability of OPLT to reduce the immunogenicity of FVIII as an antigen compared to animals administered FVIII or FVIII+OPLS. Animals were injected with FVIII, FVIII+OPLT, or FVIII+OPLS SC QW for 4 weeks. Blood samples were collected for anti-FVIII titer analysis after a one-week washout period from the last dose. The objective of the re-challenge phase of the study was to evaluate the duration of tolerance. Animals that have been successfully tolerized toward FVIII will not mount a robust immune response towards FVIII upon subsequent exposure to FVIII. After the washout period, animals were injected with (1 μg) FVIII SC QW for 2 weeks. Blood samples were collected one week after each FVIII injection for anti-FVIII titer analysis.

One week after the last dose in the treatment period, one animal from each group was selected at random for ex vivo analysis of the T-cell population. Spleens were collected and a single-cell suspension was prepared for cellular phenotyping by flow cytometry. Cells were stained and gated for CD4+ T-cells. FoxP3 expression level was evaluated for each animal.

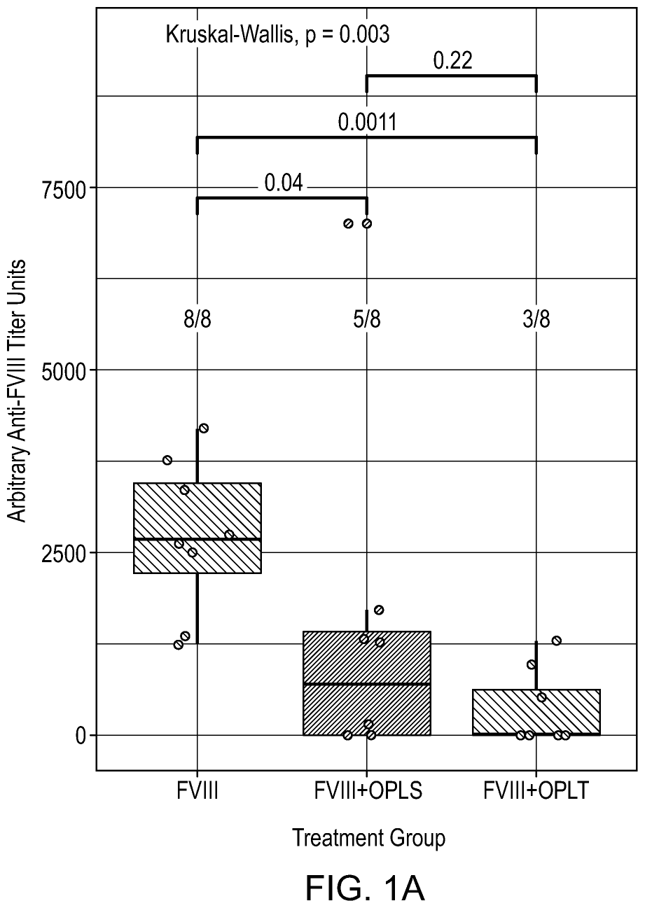

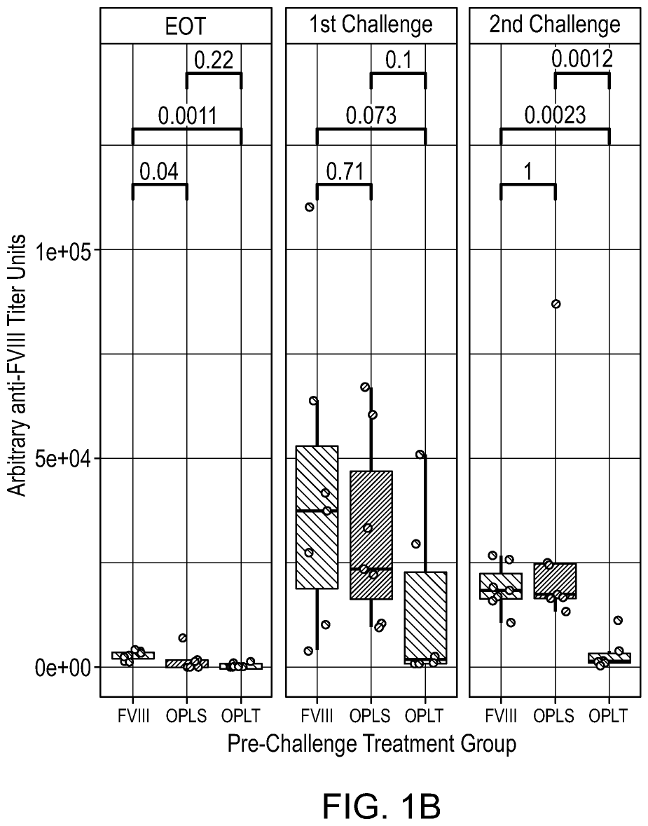

Flow cytometry analysis of splenocytes isolated from one animal in each treatment group showed an increase in FoxP3+/CD4+ T-cell in animals treated with OPLT+FVIII and OPLS+FVIII versus the mouse treated with FVIII alone (FIG. 1C). Treatment with OPLS+FVIII and OPLT+FVIII resulted in a 66% (from 2.94 to 4.89) and 188% (from 2.94 to 8.47) increase in FoxP3+/CD4+, respectively, compared to the FVIII alone group. Treatment with OPLT+FVIII resulted in a 73% increase in FoxP3+/CD4+ compared to OPLS+FVIII treatment.

Anti-FVIII titer analysis of blood samples collected after a one-week washout from the end of the treatment period (EOT) showed that all animals in the FVIII control group had developed anti-FVIII antibodies (8/8). In the OPLS+FVIII and OPLT-FVIII groups, only 5/8 and 3/8 animals developed anti-FVIII antibodies, respectively (FIG. 1A). Furthermore, titers developed in the OPLS+FVIII and the OPLT+FVIII treatment group were statistically lower than the FVIII control group (FIG. 1A).

FIG. 1B shows the anti-FVIII titer analysis of blood samples collected on weeks 1 and 2 of the re-challenge period. Animals in OPLT+FVIII treatment group did not develop a robust immune response compared to OPLS+FVIII and FVIII control animals. On average, animals in the OPLT+FVIII treatment group had lower titer than animals in the OPLS+FVIII and the FVIII control group. The statistical significance of this analysis is shown in FIG. 1B.

Anti-drug antibody formation is a major challenge for enzyme and protein replacement therapy. In this study, the ability of OPLT to reduce the immunogenicity of FVIII in animals was studied. Data presented in this study shows OPLT can reduce the immune response to FVIII in animals treated with OPLT+FVIII. This reduction in immunity is likely due to the tolerization of the immune system, as evident from the increase in FoxP3+/CD4+ T-cell as well as the reduced immune response to re-challenges with FVIII, both of which suggest immune tolerance as opposed to immune suppression. Furthermore, OPLT was more effective than OPLS in inducing sustained immunotolerization (e.g., tolerance).

### Example 2

### Reducing Immune Response to GAA in B57BL6 Mice, Comparing O-phospho-L-tyrosine (OPLT) and Liposomal O-benzyl-L-tyrosine (OBLT)

In this study, the tolerance induction properties of OPLT and liposomal OBLT were compared. Furthermore, oral tolerance and subcutaneous tolerance were evaluated using low dose liposomal OBLT. This study was conducted using acid alpha-glucosidase (GAA) as a model antigen

Recombinant human GAA is used in the treatment of Pompe disease, a life-threatening congenital lysosomal storage disease. Treatment with FDA-approved GAA is associated with a high incidence of anti-GAA antibodies. It is reported that greater than 90% of patients will develop neutralizing anti-GAA antibodies, which render therapy ineffective. Current clinical management of anti-GAA antibodies requires high doses of immunosuppressive treatment, which compromises the patient's immune system. The approach studied in this example is a non-immunosuppressive approach to addressing immunogenicity of enzyme replacement therapies such as GAA.

All animal studies were conducted in compliance with Tufts University/Tufts Medical Center & Human Nutrition Research Center on Aging. Animals were housed four in a cage, and had access to food and water ad libitum.

OPLT and OBLT were dissolved in sterile PBS, and pH was adjusted to approximately 7.5. Liposomal OBLT was prepared using DMPC. GAA in PBS was used as a control. For the tolerance induction (or treatment) period, GAA was co-mixed with OPLT or liposomal OBLT, or diluted in PBS before administration as per group assignment for each animal. For the re-challenge protocol, GAA was administered IV alone to all animals regardless of group assignment. GAA was administered with 5 mg of OPLT SC QW. Liposomal OBLT was expected to be more potent, and was administered with GAA at 50 μM (1.3 μg of OBLT/injection) SC QW or 7.2 μM (0.2 μg of OBLT/dose) PO QD. For all SC groups, GAA was dosed at 1 μg/injection (1 μg/week). For PO groups, GAA was dosed at 0.14 μg/dose QD (approximately 1 μg/week).

The objective of the treatment phase was to test the ability of OPLT or liposomal OBLT to reduce the immunogenicity of GAA, as an antigen, compared to animals administered GAA alone. Animals were injected with GAA, GAA+OPLT, or GAA+liposomal OBTL SC QW for 4 weeks. Blood samples were collected for anti-GAA analysis after a one-week washout period from the last dose. For oral tolerance, animals were administered GAA or GAA+liposomal OBLT PO, QD for 4 weeks. GAA was also administered SC QW to both groups during the treatment period. Blood samples were collected for anti-GAA titer analysis after a one-week washout period from the last dose.

The objective of the re-challenge phase of the study was to evaluate the duration of tolerance. Animals that have been successfully tolerized toward GAA will not mount a robust immune response towards GAA upon subsequent exposure to GAA. After the washout period, animals were injected with (1 μg) GAA SC QW for 2 weeks. Blood samples were collected one week after each injection for anti-GAA titer analysis.

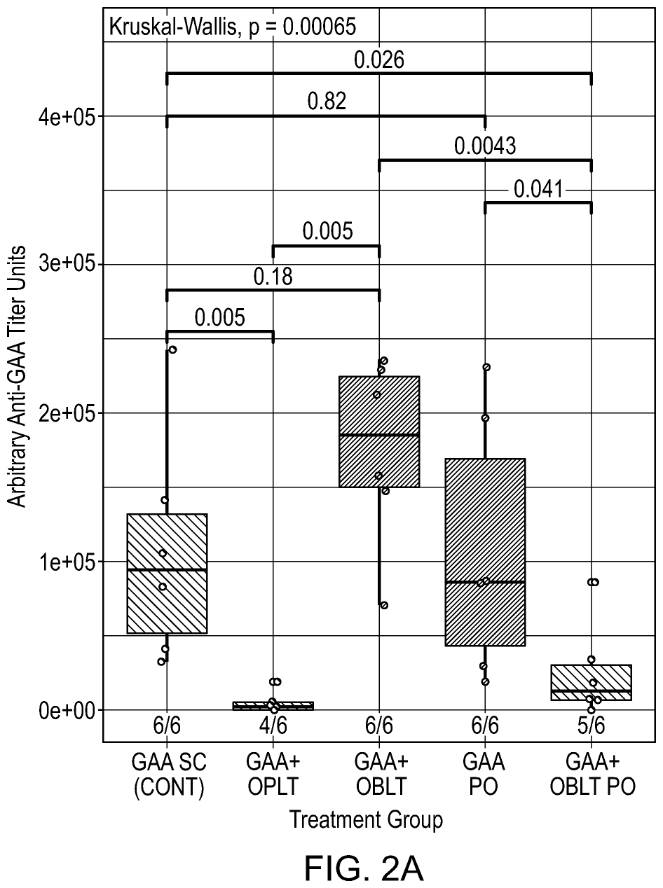

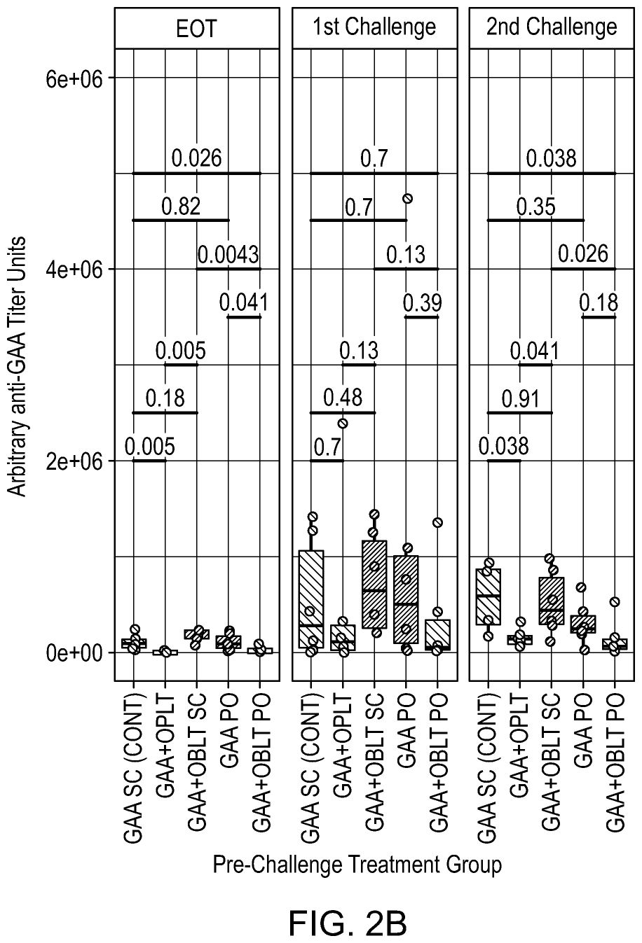

Anti-GAA titer analysis of blood samples collected after one-week washout showed that all animals in the GAA control groups (GAA SC and GAA PO) had developed anti-FVIII antibodies (6/6) (FIG. 2A). 4/6 and 5/6 animals in the treatment groups GAA-OPLT and GAA-liposomal OBLT (PO), respectively, developed titers (FIG. 2A). Animals in the GAA-liposomal OBLT SC group developed titers similar to the control groups, while titers developed in the GAA-OPLT and GAA-liposomal OBLT PO groups were statistically lower than the controls and comparators (FIG. 2A).

Anti-GAA titer analysis of blood samples collected on weeks 1 and 2 of the re-challenge period showed that animals in GAA-OPLT and GAA-liposomal OBLT PO treatment groups did not develop a robust immune response compared to GAA SC or PO groups or GAA-liposomal OBLT SC group. While this observation was not statistically significant for week one re-challenge, it was statistically significant for the week 2 re-challenge as seen in FIG. 2B.

Anti-drug antibody formation is a major challenge for enzyme and protein replacement therapy. In this study, the ability of OPLT and liposomal OBLT to reduce the immunogenicity of GAA in animals was studied. Data presented in this study show OPLT administered with GAA SC, or liposomal OBLT administered with GAA PO can reduce the immune response to GAA. This reduction in immunity is due to the tolerization of the immune system, as evident from the reduced immune response to re-challenge with GAA. This suggests immune tolerance, as opposed to immune suppression.

Furthermore, liposomal OBLT was more effective when administered orally. Interestingly, when GAA was administered orally at the same dose and frequency as GAA in the liposomal OBLT PO group, it did not result in tolerance toward GAA. This suggests that the observed oral tolerance to GAA in this study was due to the formulation of GAA with OBLT in DMPC liposomes. GAA administered SC with OBLT in a liposomal formulation was not effective when compared to OPLT or oral liposomal OBLT. This could be due to the reduced dose of OBLT compared to OPLT. This reduced dose was effective when given orally but not subcutaneously. This could suggest that the oral route is superior to SC for tolerance induction responding to lower doses of OBLT.

### Example 3

### Reducing Intolerance to AAV9 in B57BL6 Mice, Comparing O-phospho-L-tyrosine (OPLT) and Liposomal O-benzyl-L-tyrosine (OBLT)

In this study, the ability of OPLT and liposomal OBLT to induce tolerance toward AAV9 in B57BL6 mice was evaluated. Furthermore, oral tolerance versus subcutaneous tolerance was evaluated using low-dose liposomal OBLT.

All animal studies were conducted in compliance with Tufts University/Tufts Medical Center & Human Nutrition Research Center on Aging. Animals were housed four in a cage, and had access to food and water ad libitum.

OPLT and OBLT were dissolved in sterile PBS, and pH was adjusted to approximately 7.5. Liposomal OBLT was prepared using D1ViPC. AAV9 in PBS was used as a control. For treatment, AAV9 was co-mixed with OPLT or liposomal OBLT before administration, as per group assignment for each animal. AAV9 was administered with 5 mg of OPLT SC QW, 50 μg OBLT SC QW as liposomal OBLT or 7.2 μg OBLT PO QD as liposomal OBLT. For all SC groups, AAV9 was dosed at 10⁶ particles/injection. For PO groups, AAV9 was dosed at 1.43×10⁵ particles/injection.

Animals were injected with AAV9, AAV9+OPLT, or AAV9+liposomal OBLT SC QW for 4 weeks. Blood samples were collected for anti-AAV9 titer analysis after a one-week washout period from the last dose. For oral tolerance, animals were administered AAV9 or AAV9+liposomal OBLT PO, QD for 4 weeks. AAV9 was also administered SC QW to both groups during the treatment period. Blood samples were collected for anti-AAV9 titer analysis after a one-week washout period from the last dose.

One week after the last dose, one animal from each group was selected at random for ex-vivo analysis of the T-cell population. Spleens were collected and a single-cell suspension was prepared for cellular phenotyping by flow cytometry. Cells were stained and gated for CD4+ T-cells. FoxP3 expression level was evaluated for each animal.

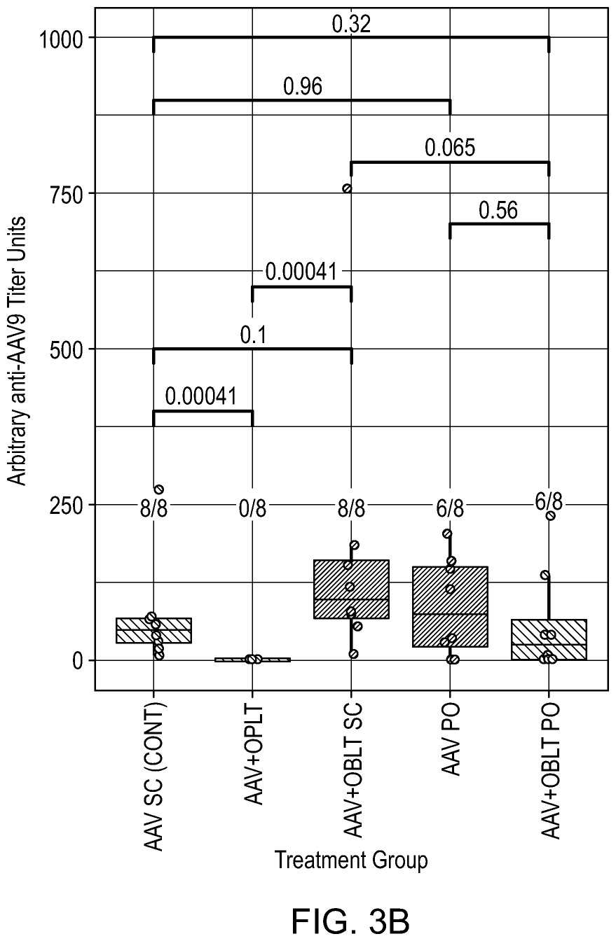

Flow cytometry analysis of splenocytes isolated from one animal in each SC treatment group showed an increase in FoxP3+/CD4+ T-cell in animals treated with OPLT+AAV9 or AAV9+liposomal OBLT compared to untreated animals. As seen in the upper panel of FIG. 3A, treatment with OPLT+AAV9 or AAV9+liposomal OBLT resulted in a 122% and 27% increase, respectively, in FoxP3+/CD4+ compared to the untreated group. In the oral group, treatment with AAV9+liposomal OBLT resulted in a 75% increase in FoxP3+/CD4+ compared to the untreated group, and 72% increase in FoxP3+/CD4+ compared to animal administered oral AAV9 alone (FIG. 3A).

Anti-AAV9 titer analysis of blood samples collected after a one-week washout showed low anti-AAV9 titer in general. All animals (8/8) in the AAV9 SC control group and AAV9+liposomal OBLT SC group developed measurable anti-AAV9 antibodies. In contrast, 0/8 animals in the AAV9+OPLT SC developed any measurable anti-AAV9 antibodies. In the oral treatment groups, 6/8 animals in each oral group developed anti-AAV9 antibodies. On average, animals treated with oral AAV9+liposomal OBLT had lower anti-AAV9 antibodies, however, the data were not statistically significant compared to control. Statistical analysis is shown in FIG. 3B

Immunogenicity is one major challenge facing the successful clinical application of gene therapy. Pre-existing antibodies to the viral particle carrying the gene therapy are a counter-indication to the administration of the gene therapy. Furthermore, nascent antibodies that result from the administration of gene therapy can impact its safety and efficacy. In this study, OPLT and liposomal OBLT were evaluated for their effects on the immunogenicity of AAV9. Subcutaneous administration of AAV9 with OPLT did not elicit an immune response compared to AAV9 administered alone.

Oral tolerance with liposomal OBLT resulted in lower anti-AAV9 antibodies on average. However, this reduction in titers was not statistically significant compared to controls. This could be due, in part, to the loading efficacy of AAV9 onto the liposomal particle compared with GAA in Example 2.

The anti-AAV9 titers correspond with the findings from the ex vivo analysis of splenocytes. Treatment with OPLT resulted in a 122% increase in FoxP3+/CD4+ T-cells. Oral administration of liposomal OBLT, however, resulted in a 75% increase in FoxP3+/CD4+ T-cells, which is less than the increase observed with OPLT. Titer analysis from the oral liposomal OBLT group was numerically less than the titers in the AAV9 oral control groups, however, this was not statistically significant. Furthermore, liposomal OBLT given by the SC route resulted in a modest 27% increase in FoxP3+/CD4+ T-cells. Titers analysis from animals in the liposomal OBLT SC group was numerically and statistically not different than control. Taken together, the data from this study and Example 1 suggest that a minimal percent increase in FoxP3+/CD4+ T-cells may be needed to obtain desired tolerogenic effects.

These in vivo data show that AAV9 formulated with OPLT does not elicit an antibody response to AAV9 after 4 weekly SC injections with AAV9, and that co-administration of AAV9 and OPLT allows for repeat dosing of AAV9. Furthermore, the data suggest that this is due to immune tolerance (e.g., Foxp3 upregulation) as opposed to immune suppression or immune ignorance. These data suggest that OPLT can enable repeat dosing with not only AAV9, but also other viral vectors/carriers (e.g., of gene therapy).

### Example 4

### Non-Specific Tolerance

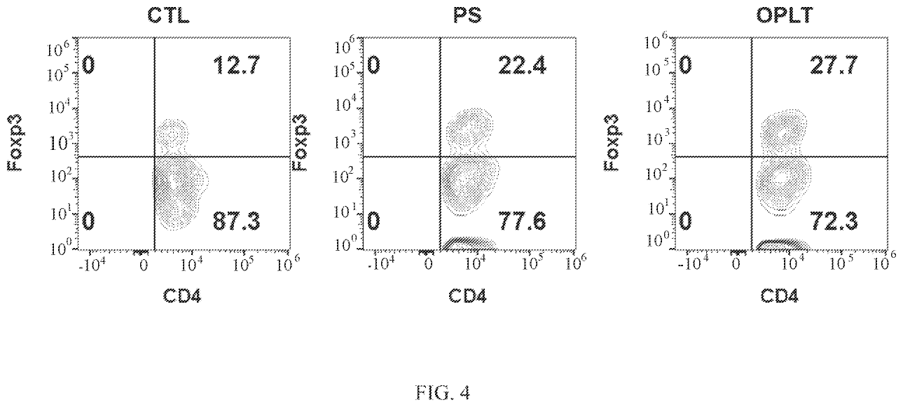

Splenocytes were cultured for three days in presence of anti-CD3/anti-CD28 in presence of 100 μM PS or 100 μM OPLT in absence of any antigen. The results of flow cytometry analysis of this experiment are shown in FIG. 4. FIG. 4 shows that treatment with OPLT can increase Foxp3+/CD4+ by 118% compared to control, versus 76% for cells treated with PS, TIM's natural ligand. The data suggest that OPLT is superior to PS in inducing Foxp3+/CD4+ in an antigen-independent manner, suggesting its potential for autoimmune disorders.

### Example 5

### Structure-Activity Relationship (SAR) Screen in RAW 264.7 Cell Line

RAW264.7 mouse monocyte macrophage cells were exposed to an increasing concentration of selected compounds in the presence of human plasma-derived FVIII as a model antigen protein. TNF-α and TGF-β production were monitored.

All molecules were formulated in RPMI media in aseptic conditions. The pH was adjusted to approximately 7.5 before dosing cells. 1,2-dimyristoyl-sn-glycero-3-phosphocholine (DM PC):PS liposomes were made at 70:30 molar ratio to make “PS” liposomes. The remaining liposomes were made by hydrating a DMPC lipid layer in PBS buffer containing a test compound to give a final molar ratio of 70:30 of DMPC:test compound.

RAW 264.7 cells were prepared, cultured, and dosed. Cells were seeded at 4×10⁵ cells/well. OPLS, OPLT and L-homocysteic acid (LHA) were dosed at 100, 85, 70, 60, 50, 30, 10, 3, 1, 0.3, 0.1, 0.03 and 0.01 mM, or at 100, 85, 70, 60, 50, 30, 10, 3 and 1 mM, as indicated. O-Benzyl-L-serine (OBLS), S-phenyl-L-cysteine (SPLC), S-benzyl-L-cysteine (SBLC) and OBLT were dosed at 100, 85, 70, 60, 50, 30, 10, 3, 1, 0.3, 0.1, 0.03, 0.01, 0.003 and 0.001 μM. Finally, 100 μl of 40 μg/ml of FVIII as a sample antigen was added to each cell. LPS and free protein were used as positive and baseline controls, respectively. TNF-α and TGF-β ELISA were conducted as per the manufacturer's instructions (R & D Systems).

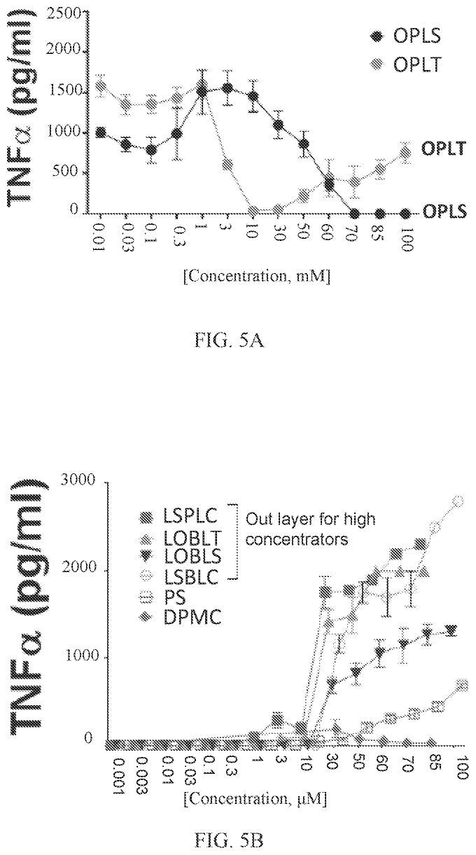

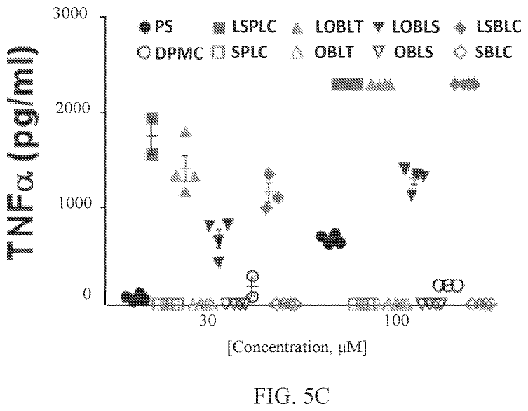

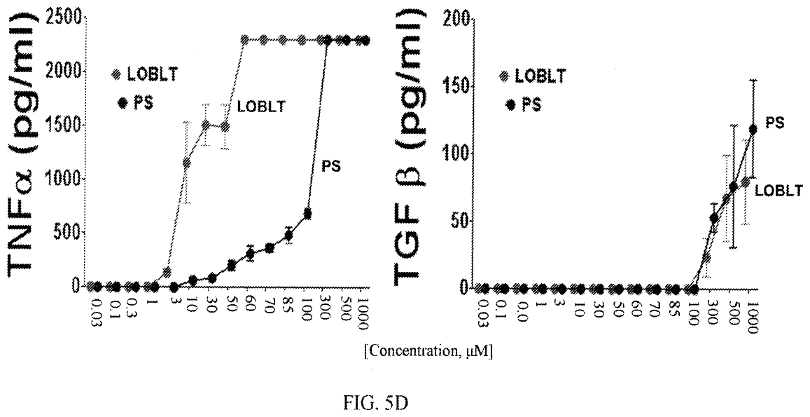

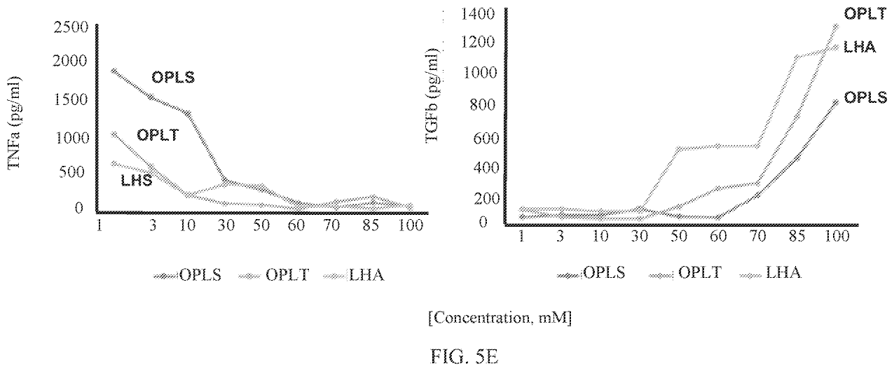

Data from ELISA showed that OPLT suppressed TNF-α production at a lower dose than OPLS (FIG. 5A). TGF-β production by OPLT mimicked that of OPLS (data not shown).

Without wishing to be bound by any particular theory, it is believed that the addition of the aromatic ring to tyrosine may play a role in the efficacy of OPLT. It may be due to increased interaction between the phenol side chain of OPLT and TIM4 opening while the increased distance between the phosphate group and the carboxylic acid allows for full engagement of the binding pocket.

In a subsequent round of screening, OBLS, SPLC, SBLC and OBLT were examined. Loading of the molecules on inert DMPC liposomes was also explored. Without wishing to be bound by any particular theory, it is believed that the lipid bilayer of the liposomes could further stabilize the interaction between the molecules and TIM4 receptors.

As seen in FIG. 5B, inert DMPC liposomes showed negligible changes in TNF-α, confirming the suitability of DMPC as an inert carrier. PS liposomes showed an increase in TNF-α, however, a plateau was not reached. The liposomal forms of SPLC, OBLT, OBLS and SBLC (LSPLC, LOBLT, LOBLS and LSBLC, respectively) all showed robust induction of TNF-α compared to PS liposomes. The free form of SPLC, OBLT, OBLS, and SBLC failed to induce TNF-α at an equivalent concentration to their liposomal forms (FIG. 5C).

Data suggest that the liposomal forms of SPLC, OBLT, OBLS, and SBLC, but not the free form, can induce TNF-α in the RAW cell line. Furthermore, induction of TNF-α by liposomal SPLC, OBLT, OBLS, and SBLC was more robust than that observed with PS liposomes. These data support the notion that a lipid bilayer may improve the interaction with the TIM4 receptor, a notion also supported by the 1,000-fold difference between the ability of OPLS and PS to induce TNF-α.

To confirm the utility of liposomal OBLT as a full agonist of TIM4, an ELISA assay comparing liposomal OBLT to PS was conducted. As shown in FIG. 5D, liposomal OBLT resulted in a more robust production of TNF-α as compared to PS liposomes, and was able to mimic the TGF-β profile of PS liposomes.

As shown in FIG. 5E, both OPLT and LHA seemed to be more potent inhibitors of TNF-α and more potent inducers of TGF-β compared to OPLS.

### Example 6

### Evaluating the Effect of OPLT on Cytokine Production by Splenocytes in Response to Different TLR Ligands

Toll-like receptors (TLRs) are key players in the innate immune response. TLRs are activated by structurally conserved molecules derived from pathogens. Once activated, TLRs trigger a cascade of events including upregulation of pro-inflammatory cytokines such as TNF-α culminating in an immune response to the pathogen.

New promising therapeutic modalities such as antisense oligonucleotides (ASOs) and gene therapy could trigger an immune reaction by activating TLR3 and/or TLR7/8, which lead to pro-inflammatory cytokine production. Commonly used immune suppressants, such as dexamethasone or rapamycin, can suppress TLR response in a nonspecific manner, resulting in a compromised immune response to pathogens. This is one of the major drawbacks for the use of such medications for the treatment of autoimmune diseases or mitigation of the immunogenicity of protein therapeutics, gene therapy, or RNA-based therapies.

Developing a therapy that can mitigate the immune response to TLR3 and/or TLR7/8 ligands such as ASOs and/or single- and double-stranded RNA while sparing the remaining TLRs could prove to be very advantageous. It has been proposed that the TIM pathway is upstream of TLR3, TLR7/8, and TLR9.

Immune modulation of the TIM pathway by OPLT was studied to assess whether OPLT could lead to modulation of TLR3, TLR7/8, and TLR9, but not TLR2 and TLR4, as those are extracellular and not cytosolic receptors.

Animals were housed four per cage, and had access to food and water ad libitum. The following materials and reagents were used in this study:

| | Provider |
|---|---|
| TNF-α ELISA Kit | |
| ssPolyU Naked | InvivoGen |
| Type A CpG oligonucleotide - | InvivoGen |
| Murine TLR9 ligand (CpG | |
| ODNs) | |
| PGN-SA, peptidoglycan | InvivoGen |
| preparation from the Gram- | |
| positive bacterium | |
| Staphylococcus aureus. | |
| LPS | |
| Polyinosinic-polycytidylic acid | InvivoGen |
| (poly(I:C)): synthetic analog of | |
| double-stranded RNA (dsRNA), | |

OPLT was dissolved in sterile PBS at 100 μM, and pH was adjusted to approximately 7.5. Splenocytes from naïve C57BL/6 mice were stained with CFSE, prepared, cultured, and dosed. Cells were seeded at 2×10⁵ cells/well.

To evaluate the effects of OPLT on FoxP3+/CD4+ T-cells, splenocytes were cultured for 3 days in presence of anti-CD3/anti-CD28 with and without 1.85 μg/ml (10 μM) OPLT. Cells were harvested at the end of a 72-hour incubation period and percent FoxP3+/CD4+ T-cells was determined by flow cytometry.

To evaluate the effects of OPLT on cytokine production by splenocytes in response to different TLR ligands, splenocytes were incubated with one of the following TLR ligands in triplicate: PGN, LPS, ODN CPG, dsRNA, or ssRNA, as per group assignment. Cells were then treated as follows:
Control: No treatment;
OPLT: Cells were dosed with 1.85 μg/ml;
Dexamethasone: Cells were dosed with 100 μg/ml;
Rapamycin: Cells were dosed with 625 μg/ml.
   Cells were incubated for 48 hours before collecting the supernatant for TNF-α analysis.

Cells were observed via a microscope at the end of the incubation period and found to be suitable for flow cytometry analysis. Treatment with 10 μM OPLT resulted in a 118% increase in FoxP3+/CD4+ T-cells versus untreated cells (27.7% versus 12.7%, respectively).

Cells were observed at the end of the induction period and did not show signs of clumping or other indications of being unhealthy. The supernatant was collected for TNF-α analysis by ELISA.

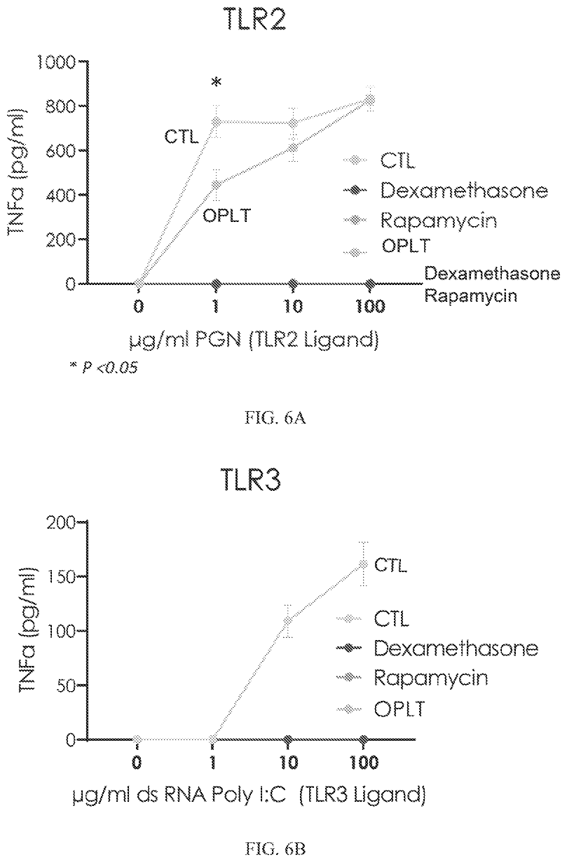

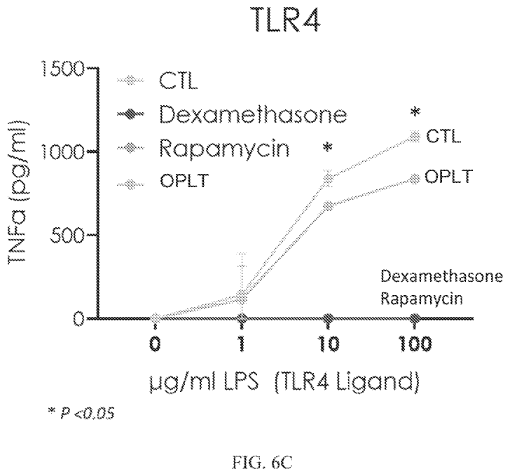

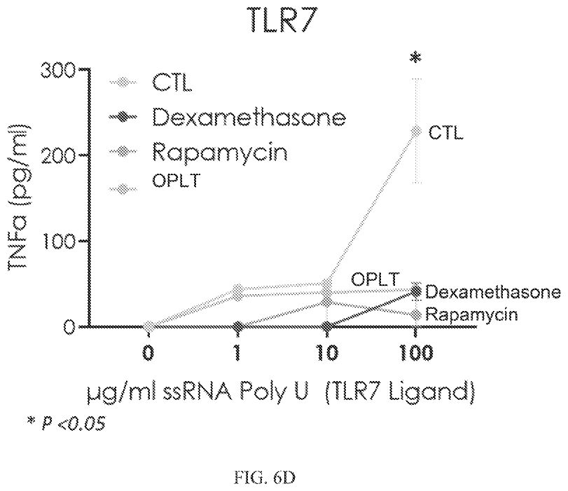

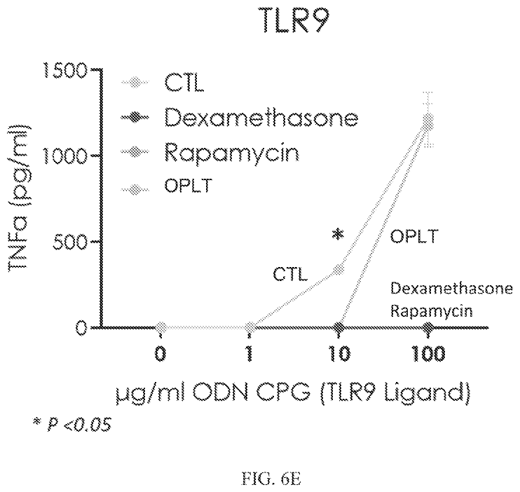

Cells incubated with peptidoglycan preparation from the gram-positive bacterium Staphylococcus aureus (PGN) showed a dose-independent increase in TNF-α production in the control group, as shown in Table 1. Treatment with dexamethasone or rapamycin abrogated splenocytes' response to the TLR2 ligand PGN. Treatment with OPLT, however, resulted in a statistically significant reduction in TNF-α in the low PGN dose group (1 ng/ml), but not the mid or high PGN dose group (FIG. 6A, Table 1).

TABLE 1
Summary statistics of changes in TNF-α in response
to increasing doses of PGN by treatment group.

| | CTL | | | Dexamethasone | | | Rapamycin | | | OPLT | | |
|---|---|---|---|---|---|---|---|---|---|---|---|---|
| PGN | mean | | | mean | | | mean | | | mean | | |
| (ng/ml) | (pg/ml) | SD | n | (pg/ml) | SD | n | (pg/ml) | SD | n | (pg/ml) | SD | n |
| 0 | 0 | 0 | 1 | 0 | 0 | 3 | 0 | 0 | 3 | 0 | 0 | 3 |
| 1 | 730 | 73 | 3 | 0 | 0 | 3 | 0 | 0 | 3 | 445 | 69 | 3 |
| 10 | 725 | 69 | 3 | 0 | 0 | 3 | 0 | 0 | 3 | 613 | 61 | 2 |
| 100 | 834 | 56 | 3 | 0 | 0 | 3 | 0 | 0 | 3 | 827 | 24 | 3 |

Cells incubated with polyinosinic-polycytidylic acid (poly(I:C)), a synthetic analog of double-stranded RNA which is a known ligand for TLR3, showed a dose-dependent increase in TNF-α production in the control group, as shown in Table 2. Treatment with dexamethasone, rapamycin, or OPLT abrogated splenocytes' response to the TLR3 ligand poly(I:C) compared to control at all doses (FIG. 6B, Table 2)

TABLE 2
Summary statistics of changes in TNF-α in response
to increasing doses of poly(I:C) by treatment group.

| | CTL | | | Dexamethasone | | | Rapamycin | | | OPLT | | |
|---|---|---|---|---|---|---|---|---|---|---|---|---|
| PolyI:C | mean | | | mean | | | mean | | | mean | | |
| (ng/ml) | (pg/ml) | SD | n | (pg/ml) | SD | n | (pg/ml) | SD | n | (pg/ml) | SD | n |
| 0 | 0 | 0 | 3 | 0 | 0 | 3 | 0 | 0 | 3 | 0 | 0 | 3 |
| 1 | 0 | 0 | 3 | 0 | 0 | 3 | 0 | 0 | 3 | 0 | 0 | 3 |
| 10 | 109 | 15 | 3 | 0 | 0 | 3 | 0 | 0 | 3 | 0 | 0 | 3 |
| 100 | 162 | 20 | 3 | 0 | 0 | 3 | 0 | 0 | 3 | 0 | 0 | 3 |

Cells incubated with lipopolysaccharides (LPS), a known TLR4 ligand, showed a dose-dependent increase in TNF-α production in the control group, as shown in Table 3. Treatment with dexamethasone or rapamycin abrogated splenocytes' response to the TLR4 ligand LPS compared to control at all doses (FIG. 6C and Table 3). Treatment with OPLT, however, did not abrogate splenocytes' response to LPS. Despite a statistically significant reduction in TNF-α in the mid- and high-dose LPS groups treated with 10 μM OPLT, OPLT did not completely abrogate the response to LPS, as did dexamethasone and rapamycin (FIG. 6C and Table 3).

TABLE 3
Summary statistics of changes in TNF-α in response
to increasing doses of LPS by treatment group.

| | CTL | | | Dexamethasone | | | Rapamycin | | | OPLT | | |
|---|---|---|---|---|---|---|---|---|---|---|---|---|
| LPS | mean | | | mean | | | mean | | | mean | | |
| (ng/ml) | (pg/ml) | SD | n | (pg/ml) | SD | n | (pg/ml) | SD | n | (pg/ml) | SD | n |
| 0 | 0 | 0 | 3 | 0 | 0 | 3 | 0 | 0 | 3 | 0 | 0 | 3 |
| 1 | 143 | 247 | 3 | 0 | 0 | 3 | 0 | 0 | 3 | 116 | 201 | 3 |
| 10 | 840 | 50 | 3 | 0 | 0 | 3 | 0 | 0 | 3 | 676 | 21 | 3 |
| 100 | 1091 | 33 | 3 | 0 | 0 | 3 | 0 | 0 | 3 | 838 | 13 | 3 |

Cells incubated with ssPolyU, a known TLR7 ligand, showed a dose-dependent increase in TNF-α production in the control group, as shown in Table 4. Treatment with dexamethasone, rapamycin, or OPLT reduced splenocytes' response to a high dose of the TLR7 ligand Poly U compared to control, and this effect was statistically significant (FIG. 6D). At the mid and low doses of Poly U, dexamethasone, rapamycin, and OPLT treatment resulted in equivalent response to the control (FIG. 6D and Table 4).

TABLE 4
Summary statistics of changes in TNF-α in response
to increasing doses of PolyU by treatment group.

| | CTL | | | Dexamethasone | | | Rapamycin | | | OPLT | | |
|---|---|---|---|---|---|---|---|---|---|---|---|---|
| Poly U | mean | | | mean | | | mean | | | mean | | |
| (ng/ml) | (pg/ml) | SD | n | (pg/ml) | SD | n | (pg/ml) | SD | n | (pg/ml) | SD | n |
| 0 | 0 | 0 | 1 | 0 | 0 | 3 | 0 | 0 | 3 | 0 | 0 | 3 |
| 1 | 44 | 4 | 3 | 0 | 0 | 1 | 0 | 0 | 1 | 36 | 0 | 1 |
| 10 | 51 | 4 | 3 | 0 | 0 | 2 | 29 | 25 | 3 | 40 | 9 | 3 |
| 100 | 229 | 61 | 3 | 41 | 10 | 2 | 14 | 24 | 3 | 43 | 1 | 3 |

Cells incubated with type A CpG oligonucleotide (ODN CpG), a known TLR9 ligand, showed a dose-dependent increase in TNF-α production in the control group, as shown in Table 5. Treatment with dexamethasone, rapamycin, or OPLT reduced splenocytes' response to mid-dose of the TLR9 ligand ODN CpG compared to control, and this effect was statistically significant (FIG. 6E). At the high dose of ODN CpG, dexamethasone and rapamycin suppressed splenocytes' response, but OPLT did not (FIG. 6E and Table 5).

TABLE 5
Summary statistics of changes in TNF-α in response
to increasing doses of ODN CPG by treatment group.

| | CTL | | | Dexamethasone | | | Rapamycin | | | OPLT | | |
|---|---|---|---|---|---|---|---|---|---|---|---|---|
| ODN CpG | mean | | | mean | | | mean | | | mean | | |
| (ng/ml) | (pg/ml) | SD | n | (pg/ml) | SD | n | (pg/ml) | SD | n | (pg/ml) | SD | n |
| 0 | 0 | 0 | 1 | 0 | 0 | 3 | 0 | 0 | 3 | 0 | 0 | 3 |
| 1 | 0 | 0 | 3 | 0 | 0 | 3 | 0 | 0 | 3 | 0 | 0 | 3 |
| 10 | 338 | 6 | 2 | 0 | 0 | 3 | 0 | 0 | 3 | 0 | 0 | 3 |
| 100 | 1220 | 153 | 3 | 0 | 0 | 3 | 0 | 0 | 3 | 1178 | 126 | 3 |

The ability of TLRs to recognize and respond to molecular pathogenic patterns, such as bacterial cell wall components and single- and double-stranded RNA, is an important step in initiating an immune response to pathogens. ASO and gene therapy, which aim to deliver therapeutic material, may share a common structure as TLR ligands, thereby promoting an immune response, even where one is not desired.

By specifically targeting TIM3, it was hypothesized that it would be possible to modulate the immune response from cytosolic TLRs (TLR3, TLR7/8, and TLR9) but not the cellular membrane-bound TLR2 and TLR4. This is because TIM3 is upstream of TLR3, TLR7/8, and TLR9.

In this study, the effect of a therapeutically relevant concentration of OPLT on cytokine production by splenocytes in response to different TLR ligands was evaluated. It has previously been shown that a doubling of FoxP3+/CD4+ T-cells in vivo is correlated with reduced immunogenicity of co-administered biologics. Here, it was confirmed that a 10 μM dose of OPLT results in the desired increased in FoxP3+/CD4+ T-cells ex vivo. Indeed, 10 μM OPLT resulted in a 118% increase in FoxP3+/CD4+ T-cells, justifying the use of this dose in evaluating the effect of OPLT on cytokine production by TLR ligands.

Unlike dexamethasone and rapamycin, 10 μM OPLT did not abrogate the cytokine response of splenocytes stimulated ex vivo with TLR2 and TLR4 ligands. However, 10 μM OPLT was as effective as dexamethasone and rapamycin in reducing or eliminating the cytokine response of splenocytes stimulated ex vivo with two cytosolic TLR ligands, TLR3 and TLR7 ligands.

For the cytosolic TLR9, the response to ODN CpG in the presence of 10 μM OPLT was nuanced. OPLT was as effective as dexamethasone and rapamycin when splenocytes were exposed to the mid dose of ODN CpG; however, at the high dose of ODN CpG, OPLT did not affect cytokine response.

Collectively, these data support the hypothesis that OPLT modulates the immune response to some, but not all, TLR ligands, unlike general immune-suppressive agents. Specifically, the data show that dexamethasone, rapamycin, and OPLT treatment decreased TNFα production in the presence of the TLR ligands PolyI:C (TLR3), PolyU (TLR7/8) or ODN CpG (TLR9) in comparison to the control group. However, unlike dexamethasone and rapamycin, OPLTs did not decrease TNFα production in the presence of TLR ligands PGM (TLR2) and LPS (TLR4). OPLT mitigated cytokine production by TLR3, 7/8, and 9 but not TLR2 or TLR4 in vitro, suggesting that OPLT does not function as a general immune suppressive agent but rather as an immune modulator.

OPLT can induce tolerogenic signals and reduce immunogenicity to biologics and the cytokine response to single- and double-strand RNA, but not other danger signals. This makes OPLT an attractive candidate for further development as an immune-modulatory agent to reduce the immunogenicity of biologics, gene therapy, and ASOs.

### Example 7

### Additional Compounds Upregulate Foxp3+/CD4+ T-Cells

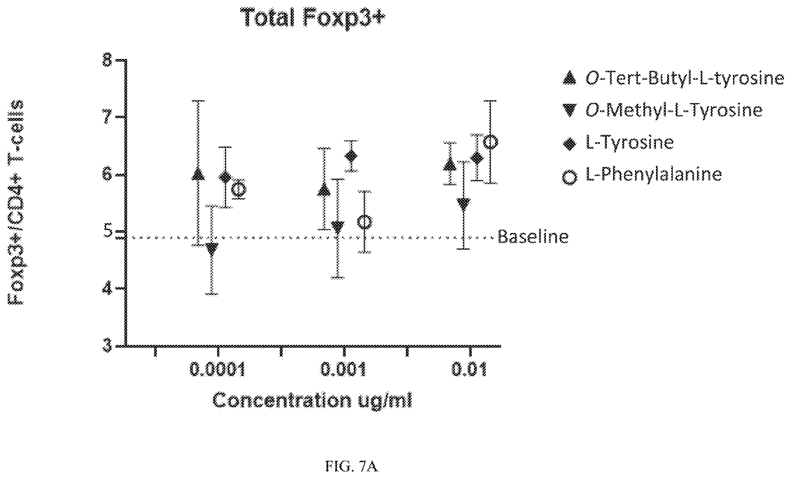

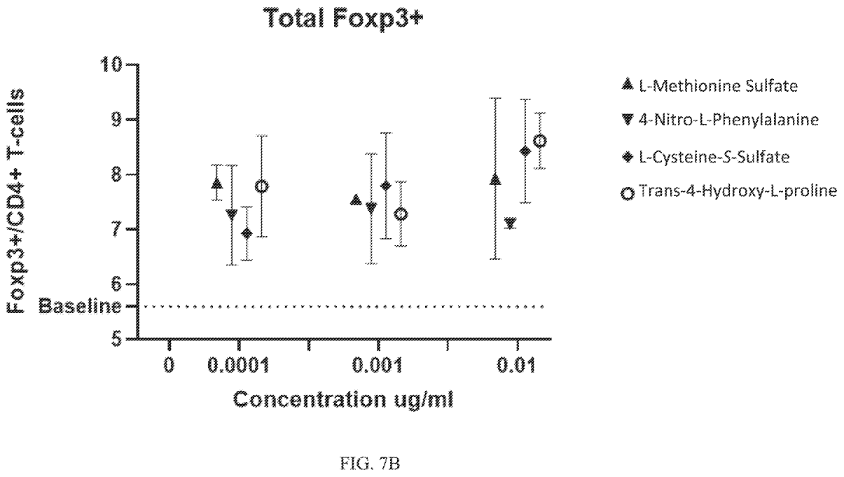

Human peripheral blood mononuclear cells (PBMC) were isolated. Isolated cells were plated at a density of 2×10⁵ cells/well, and dosed with 0.0001, 0.001 or 0.01 ng/mL of O-tert-butyl-L-tyrosine, O-methyl-L-tyrosine, L-tyrosine, L-phenylalanine, L-methionine sulfate, 4-nitro-L-phenylalanine, L-cysteine-S-sulfate or trans-4-hydroxy-L-proline in triplicate at least. At least three wells were left untreated, and used for a baseline. After five days of culture, cells were stained for CD3, CD4 and Foxp3, and analyzed by flow cytometry. The percent of CD4+/Foxp3+ T-cells were plotted, and the results are shown in FIGS. 7A and 7B.

The data depicted in FIGS. 7A and 7B show that the tested compounds desirably upregulate Foxp3+/CD4+ T-cells with varying levels of potency.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of immunotolerizing a subject in need thereof to an antigen, comprising administering to the subject a therapeutically effective amount of a composition comprising an amino acid, or a pharmaceutically acceptable salt thereof, wherein:
(i) the amino acid is a hydroxyamino acid or thioamino acid; and
the hydroxyl of the hydroxyamino acid or the thiol of the thioamino acid, respectively, is substituted with -L(R)ₙ;
L is absent, or a (C₁-C₈)alkyl or (C₂-C₈)alkenyl optionally substituted with one or more fluoro;
each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2; or
(ii) the amino acid is tyrosine; and
the hydroxyl of the tyrosine is substituted with (C₁-C₈)alkyl or (C₂-C₈)alkenyl optionally substituted with one or more fluoro; or
the hydroxyl of the tyrosine is replaced with —H; or
(iii) the amino acid is a hydroxyamino acid or thioamino acid; and
the hydroxyl of the hydroxyamino acid or the thiol of the thioamino acid, respectively, is substituted or replaced with —PO₃H₂, —SO₃H or —NO₂; or
(iv) the amino acid is a naturally-occurring hydroxyamino acid or thioamino acid, provided the amino acid is not O-phospho-L-serine.

2. A method of inhibiting or reducing an antigen-specific antibody titer in a subject, comprising administering to the subject a therapeutically effective amount of a composition comprising an amino acid, or a pharmaceutically acceptable salt thereof, wherein:
(i) the amino acid is a hydroxyamino acid or thioamino acid; and
the hydroxyl of the hydroxyamino acid or the thiol of the thioamino acid, respectively, is substituted with -L(R)ₙ;
L is absent, or a (C₁-C₈)alkyl or (C₂-C₈)alkenyl optionally substituted with one or more fluoro;
each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2; or
(ii) the amino acid is tyrosine; and
the hydroxyl of the tyrosine is substituted with (C₁-C₈)alkyl or (C₂-C₈)alkenyl optionally substituted with one or more fluoro; or
the hydroxyl of the tyrosine is replaced with —H; or
(iii) the amino acid is a hydroxyamino acid or thioamino acid; and
the hydroxyl of the hydroxyamino acid or the thiol of the thioamino acid, respectively, is substituted or replaced with —PO₃H₂, —SO₃H or —NO₂; or
(iv) the amino acid is a naturally-occurring hydroxyamino acid or naturally-occurring thioamino acid,
provided the amino acid is not O-phospho-L-serine.

3. The method of claim 1 or 2, further comprising administering the antigen, or an immunogenic fragment thereof, to the subject.

4. The method of any one of claims 1-3, wherein the antigen, or an immunogenic fragment thereof, and the composition are co-administered.

5. The method of any one of claims 1-4, wherein the composition comprises the amino acid, or a pharmaceutically acceptable salt thereof, and the antigen, or an immunogenic fragment thereof.

6. The method of any one of claims 1-5, wherein the antigen is a protein.

7. The method of any one of claims 1-6, wherein the antigen is a self-antigen.

8. The method of any one of claims 1-6, wherein the antigen is a foreign antigen.

9. The method of claim 8, wherein the foreign antigen is an antigenic therapy.

10. The method of claim 8 or 9, wherein the foreign antigen is a therapeutic protein.

11. The method of any one of claims 8-10, wherein the foreign antigen is an enzyme replacement therapy.

12. The method of claim 8 or 9, wherein the foreign antigen is a cellular or gene therapy.

13. A method of inducing a population of regulatory T-cells or increasing the activity or level of tolerogenic T-cells in a subject, comprising administering to the subject a therapeutically effective amount of a composition comprising an amino acid, or a pharmaceutically acceptable salt thereof, wherein:
(i) the amino acid is a hydroxyamino acid or thioamino acid; and
the hydroxyl of the hydroxyamino acid or the thiol of the thioamino acid, respectively, is substituted with -L(R)ₙ;
L is absent, or a (C₁-C₈)alkyl or (C₂-C₈)alkenyl optionally substituted with one or more fluoro;
each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2; or
(ii) the amino acid is tyrosine; and
the hydroxyl of the tyrosine is substituted with (C₁-C₈)alkyl or (C₂-C₈)alkenyl optionally substituted with one or more fluoro; or
the hydroxyl of the tyrosine is replaced with —H; or
(iii) the amino acid is a hydroxyamino acid or thioamino acid; and
the hydroxyl of the hydroxyamino acid or the thiol of the thioamino acid, respectively, is substituted or replaced with —PO₃H₂, —SO₃H or —NO₂; or
(iv) the amino acid is a naturally-occurring hydroxyamino acid or naturally-occurring thioamino acid,
provided the amino acid is not O-phospho-L-serine.

14. A method of treating an autoimmune disorder in a subject in need thereof, comprising administering to the subject a therapeutically effective amount of a composition comprising an amino acid, or a pharmaceutically acceptable salt thereof, wherein:
(i) the amino acid is a hydroxyamino acid or thioamino acid; and
the hydroxyl of the hydroxyamino acid or the thiol of the thioamino acid, respectively, is substituted with -L(R)ₙ;
L is absent, or a (C₁-C₈)alkyl or (C₂-C₈)alkenyl optionally substituted with one or more fluoro;
each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2; or
(ii) the amino acid is tyrosine; and
the hydroxyl of the tyrosine is substituted with (C₁-C₈)alkyl or (C₂-C₈)alkenyl optionally substituted with one or more fluoro; or
the hydroxyl of the tyrosine is replaced with —H; or
(iii) the amino acid is a hydroxyamino acid or thioamino acid; and
the hydroxyl of the hydroxyamino acid or the thiol of the thioamino acid, respectively, is substituted or replaced with —PO₃H₂, —SO₃H or —NO₂; or
(iv) the amino acid is a naturally-occurring hydroxyamino acid or naturally-occurring thioamino acid,
provided the amino acid is not O-phospho-L-serine.

15. The method of claim 14, wherein the autoimmune disorder is achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II and III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenic purpura (TTP), thyroid eye disease (TED), Tolosa-Hunt syndrome (THS), transverse myelitis, Type 1 diabetes, ulcerative colitis (UC), undifferentiated connective tissue disease (UCTD), uveitis, vasculitis, vitiligo or Vogt-Koyanagi-Harada Disease.

16. The method of claim 14 or 15, further comprising administering a self-antigen associated with the autoimmune disorder, or an immunogenic fragment thereof, to the subject.

17. The method of claim 16, wherein the self-antigen, or an immunogenic fragment thereof, and the composition are co-administered.

18. The method of claim 16 or 17, wherein the composition comprises the amino acid, or a pharmaceutically acceptable salt thereof, and the self-antigen, or an immunogenic fragment thereof.

19. A method of treating a disease, disorder or condition in a subject in need thereof with an antigenic therapy, comprising administering to the subject the antigenic therapy and, in an amount sufficient to immunotolerize the subject to the antigenic therapy, a composition comprising an amino acid, or a pharmaceutically acceptable salt thereof, wherein:
(i) the amino acid is a hydroxyamino acid or thioamino acid; and
the hydroxyl of the hydroxyamino acid or the thiol of the thioamino acid, respectively, is substituted with -L(R)ₙ;
L is absent, or a (C₁-C₈)alkyl or (C₂-C₈)alkenyl optionally substituted with one or more fluoro;
each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2; or
(ii) the amino acid is tyrosine; and
the hydroxyl of the tyrosine is substituted with (C₁-C₈)alkyl or (C₂-C₈)alkenyl optionally substituted with one or more fluoro; or
the hydroxyl of the tyrosine is replaced with —H; or
(iii) the amino acid is a hydroxyamino acid or thioamino acid; and
the hydroxyl of the hydroxyamino acid or the thiol of the thioamino acid, respectively, is substituted or replaced with —PO₃H₂, —SO₃H or —NO₂; or
(iv) the amino acid is a naturally-occurring hydroxyamino acid or naturally-occurring thioamino acid,
provided the amino acid is not O-phospho-L-serine.

20. The method of claim 19, wherein the antigenic therapy and the composition are co-administered.

21. The method of claim 19 or 20, wherein the composition comprises the amino acid, or a pharmaceutically acceptable salt thereof, and the antigenic therapy.

22. The method of any one of claims 19-21, further comprising administering to the subject a therapeutically effective amount of the antigenic therapy in the absence of the composition.

23. The method of any one of claims 19-22, wherein the antigenic therapy is a gene therapy.

24. The method of any one of claims 19-22, wherein the antigenic therapy is a protein replacement therapy.

25. The method of any one of claims 1-24, further comprising administering to the subject an additional therapeutic agent.

26. The method of any one of claims 1-25, wherein the composition is administered orally.

27. The method of any one of claims 1-25, wherein the composition is administered subcutaneously.

28. The method of any one of claims 1-27, wherein the composition comprises a plurality of lipid particles comprising one or more lipids, or a pharmaceutically acceptable salt thereof, and the amino acid, or a pharmaceutically acceptable salt thereof.

29. The method of claim 28, wherein the lipid particles are in the form of liposomes.

30. The method of claim 28 or 29, wherein the one or more lipids includes a phospholipid, or a pharmaceutically acceptable salt thereof.

31. The method of claim 30, wherein the phospholipid is a saturated phospholipid, or a pharmaceutically acceptable salt thereof.

32. The method of claim 31, wherein the phospholipid is dimyristoylphosphatidylcholine

0. MPC) or 1,2-distearoyl-sn-glycero-3-phosphocholine 18:0 (DSPC).

33. The method of any one of claims 28-32, wherein the molar percentage of the amino acid, or a pharmaceutically acceptable salt thereof, in the lipid particle is from about from about 1% to about 50%.

34. The method of any one of claims 28-33, wherein the molar percentage of the one or more lipids, or a pharmaceutically acceptable salt thereof, in the lipid particle is from about from about 50% to about 99%.

35. The method of any one of claims 28-34, wherein the lipid particles further comprise the antigen, or an immunogenic fragment thereof, self-antigen, or an immunogenic fragment thereof, or antigenic therapy.

36. The method of any one of claims 1-35, wherein the composition further comprises a pharmaceutically acceptable carrier.

37. The method of any one of claims 1-36, wherein the amino acid is a naturally-occurring amino acid.

38. The method of any one of claims 1-37, wherein the amino acid is a L-amino acid.

39. The method of any one of claims 1-38, wherein the hydroxyamino acid or thioamino acid is serine, threonine, cysteine, homocysteine, tyrosine or hydroxyproline.

40. The method of any one of claims 1-39, wherein:
the amino acid is a hydroxyamino acid or thioamino acid; and
the hydroxyl of the hydroxyamino acid or the thiol of the thioamino acid, respectively, is substituted with -L(R)ₙ;
L is absent, or a (C₁-C₈)alkyl or (C₂-C₈)alkenyl optionally substituted with one or more fluoro;
each R is independently a (C₆-C₁₅)aryl or (C₅-C₁₅)heteroaryl optionally substituted with one or more substituents independently selected from halo, (C₁-C₃)alkoxy, (C₁-C₃)haloalkoxy, (C₁-C₃)alkyl or (C₁-C₃)haloalkyl; and
n is 1 or 2.

41. The method of claim 40, wherein the amino acid is serine, threonine, cysteine, homocysteine or tyrosine.

42. The method of claim 40 or 41, wherein L is absent or (C₁-C₃)alkyl optionally substituted with one or more fluoro.

43. The method of any one of claims 40-42, wherein each R is independently a (C₆-C₁₅)aryl optionally substituted with one or more substituents independently selected from halo, (C₁-C₃)alkoxy, (C₁-C₃)haloalkoxy, (C₁-C₃)alkyl or (C₁-C₃)haloalkyl.

44. The method of any one of claims 40-43, wherein n is 1.

45. The method of any one of claims 40-44, wherein -L(R)ₙ is phenyl or benzyl.

46. The method of any one of claims 1-38 wherein the amino acid is tyrosine; and the hydroxyl of the tyrosine is substituted with (C₁-C₈)alkyl or (C₂-C₈)alkenyl optionally substituted with one or more fluoro; or the hydroxyl of the tyrosine is replaced with —H.

47. The method of any one of claims 1-39, wherein the amino acid is a hydroxyamino acid or thioamino acid; and the hydroxyl of the hydroxyamino acid or the thiol of the thioamino acid, respectively, is substituted or replaced with —PO₃H₂, —SO₃H or —NO₂.

48. The method of claim 47, wherein the amino acid is a hydroxyamino acid or thioamino acid, wherein the hydroxyl of the hydroxyamino acid is substituted with —PO₃H₂; and the thiol of the thioamino acid is replaced with —SO₃H.

49. The method of claim 47 or 48, wherein the amino acid is threonine, cysteine, homocysteine or tyrosine.

50. The method of any one of claims 1-39, wherein the amino acid is a naturally-occurring hydroxyamino acid or naturally-occurring thioamino acid.

51. The method of any one of claims 1-39, wherein the amino acid is L-homocysteic acid, O-phospho-L-tyrosine or 4-nitro-L-phenylalanine, or represented by one of the following structural formulas: 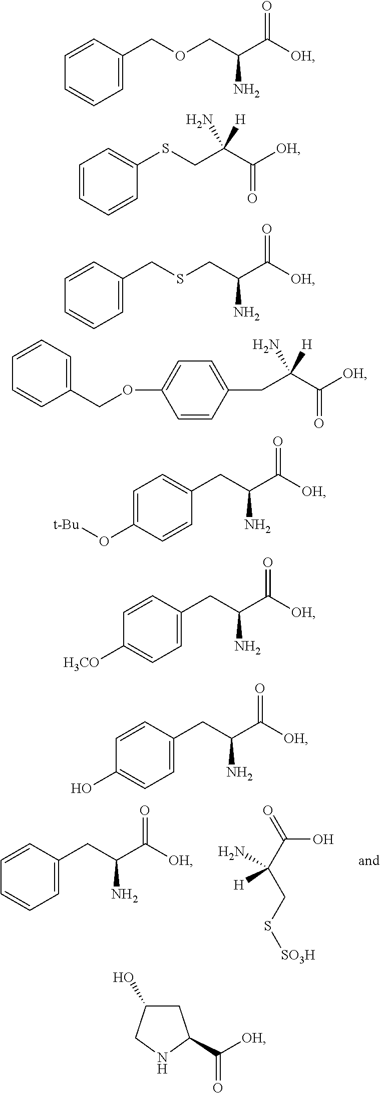
or a pharmaceutically acceptable salt of any of the foregoing.

52. The method of claim 51, wherein the amino acid is represented by one of the following structural formulas: 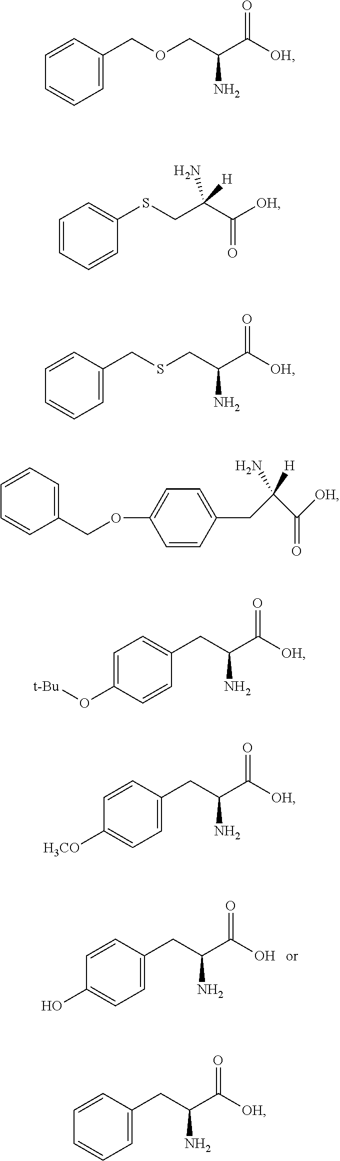
or a pharmaceutically acceptable salt of the foregoing.

53. The method of claim 51, wherein the amino acid is L-homocysteic acid, O-phospho-L-tyrosine, 4-nitro-L-phenylalanine, L-cysteine-S-sulfate or trans-4-hydroxy-L-proline, or a pharmaceutically acceptable salt of the foregoing.
